(19) United States
(12) Patent Application Publication
(10) Pub. No.: US 2025/0121212 A1
(43) Pub. Date: Apr. 17, 2025

(54) METHOD TO SYNTHESIZE ALGINATE-BASED POROUS HYDROGEL MATERIAL WITH MECHANICAL AND RADIOLOGICAL PROPERTIES EQUIVALENT TO HUMAN ORGAN

(71) Applicant: UNIVERSITY OF CENTRAL FLORIDA RESEARCH FOUNDATION, INC., Orlando, FL (US)

(72) Inventors: Jihua GOU, Orlando, FL (US); Olusegun ILEGBUSI, Orlando, FL (US); Xiangpeng LI, Orlando, FL (US)

(21) Appl. No.: 18/914,885

(22) Filed: Oct. 14, 2024

Related U.S. Application Data
(60) Provisional application No. 63/543,801, filed on Oct. 12, 2023.

(51) Int. Cl. A61N 5/10; G09B 23/30

(52) U.S. Cl. CPC A61N 5/1075; G09B 23/30; A61N 2005/1076

(57) ABSTRACT

Disclosed is a hydrogel and hydrogel-based foam, which are made of a naturally-derived material, alginate hydrogel in combination with CaCO₃, glucono delta-lactone (GDL), and sodium lauryl ether sulfate, as well as methods for making alginate hydrogel and alginate hydrogel foam, which have radiological and mechanical properties equivalent to those of human lung. The material is environment-friendly, and the hydrogel properties can be readily adjusted by changing the alginate concentration, Ca²⁺ ratio, and solution volume in the container. The hydrogel and hydrogel foam disclosed here have potential to be used for making tissue-equivalent phantoms of various human organs, in particular human lung, for radiotherapy dosimetry, and other applications such as bioimplants, artificial biological tissues, soft robotics, and optics.

### BACKGROUND

Radiation therapy is a common cancer treatment method [1]. Motion of organs and tissues often cause X-ray localization error during radiotherapy [2], which may result in under-treatment of tumor and over-exposure of surrounding healthy tissues to unnecessary radiation. Therefore, it is imperative to be able to predict the motion of organs and tissues to assist in minimization of tumor localization errors. Here, the lung is the targeted organ due to the inherent large deformation and movement when the patient breathes during radiotherapy. Several finite element models have been developed and applied to simulate the movement of human lung [3-5]. However, human organs and tissues have complex geometries and are highly patient-specific. Therefore, materials with tissue-equivalent properties, both mechanical and radiological, are desirable for the development of physical phantoms for validation of theoretical models, assist better medical treatment as well as for medical training purposes.

Lung tissue is assumed as a uniform elastic material. The density (ρ) of human lung tissue is reported to be 1.06 g/cm³, and the density of inflated lung is 0.26 g/cm³ [6]. A representative Poisson's ratio of lung is 0.43 [7]. A wide range of Young's modulus (YM) of lung tissue has been reported from application of different experimental methods. Lai-Fook et al. [8] measured the YM of human lung to be 0.42-6.72 kPa under uniform inflation. Goss et al. [9] reported the YM to be 2.17 kPa applying magnetic resonance elastography. Liu et al. [10] reported it to be 0.03-57.2 kPa through direct measurement. Ilegbusi et al. [11] estimated the YM to be 0.1-2.7 kPa according to the 4D CT scan data from specific patients.

For the radiological properties of lung, a few parameters were used to describe the interaction behavior of lung tissue with X-ray, including elemental composition, electron density (ρₑ), effective atomic number (Z_{eff}), mean excitation energy (I), mass attenuation coefficients (μ/ρ) in which μ is the linear attenuation coefficient and ρ is the density and Hounsfield Unit (HU), which is directly related to μ/ρ.

The electron density ρₑ is defined as the number of electrons per unit volume and calculated by Equation (1):

$${ρ}_{e} = ρ {N}_{A} \text{?} \frac{{w}_{i} {Z}_{i}}{{A}_{i}} \qquad ( 1 )$$

$$\text{?} \text{indicates text missing or illegible when filed}$$

where ρ is the density of the material in g/cm³, NA is the Avogadro constant, wᵢ is the weight fraction, Zᵢ is the atomic number, and Aᵢ is the atomic weight of the i^{th} element.

Atomic numbers are directly related to the nature of radiation interaction within the material. For compounds and mixtures which include more than one element, such as tissue, an effective atomic number Z_{eff} needs to be calculated. One proposed formula is obtained from Murty et al. [12]:

$${Z}_{eff} = \sqrt[\text{?}]{\text{?}} \qquad ( 2 )$$

$$\text{?} \text{indicates text missing or illegible when filed}$$

where αᵢ is the fractional number of electrons belonging to the i^{th} element.

The mean excitation energy I describes how easily the targeted material absorbs the kinetic energy from the particle penetrating the material. It describes the property of the targeted material only and is independent of the properties of the particles. I can be calculated by applying Bragg additivity rule:

$$ln ( I ) = \frac{\text{?} {w}_{i} ( \frac{{Z}_{i}}{{A}_{i}} ) ln ( {I}_{i} )}{\text{?} {w}_{i} ( \frac{{Z}_{i}}{{A}_{i}} )} \qquad ( 3 )$$

$$\text{?} \text{indicates text missing or illegible when filed}$$

where Iᵢ is the excitation energy of the i^{th} element.
μ/ρ describes the penetration and energy deposition by photons in the targeted materials. It can be calculated by:

$$\frac{μ}{ρ} = {x}^{- 1} ln ( \frac{I}{{I}_{0}} ) \qquad ( 4 )$$

where x is the thickness of the material, I₀ is the incident intensity, I is the intensity of the photons after penetrating the material. The data for μ/ρ is obtained directly from NIST Standard Reference Database 126 [13]. The values of μ/ρ for mixtures and compounds are obtained according to the rule of simple additivity, thus:

$$\frac{μ}{ρ} = \text{?} {w}_{i} ( \frac{μ}{ρ} \text{?} \qquad ( 5 )$$

$$\text{?} \text{indicates text missing or illegible when filed}$$

The Hounsfield Unit HU is a standardized dimensionless unit used in computed tomography (CT) to measure the radiodensity, which is linearly transformed from the measured attenuation coefficients μ. It is defined as:

$$HU = 1000 \times \frac{μ - {μ}_{water}}{{μ}_{water} - {μ}_{air}} \qquad ( 6 )$$

where μ_{water} is the attenuation coefficient of water and μₐᵢᵣ is the attenuation coefficient of air. Therefore, it can be concluded from Equation (6) that the HU value of water is 0 and HU value of air is −1000. The HU value of human lung is in the range −600 to −700 [14].

In order to develop soft and flexible materials with desired properties, there are several material requirements that should be met, including the density ρ, elasticity (for example, Young's Modulus, YM), and radiological properties. The density ρ is a basic property for materials, especially for materials mimicking organs and tissues. It influences many aspects of material behaviors. The value of ρ for most human organs/tissues is about 1 g/cm³, which is close to the density of water [15]. Elasticity is the key factor governing the deformation behavior of materials under external load. Different organs and tissues have different elasticity values. However, most of the organs and tissues have a relatively low elasticity. For example, the YM of human lung is in the range 1-13 kPa. It is vital that the elasticity of substitute material be consistent with human tissues. Another important property is the radiological property, which is directly related to the accuracy of radiation absorption behavior of tissue-equivalent materials. The radiological properties of interest include ρₑ, Z_{eff}, I, and μ/ρ. Currently, a series of polyurethane (PU) foam-based tissue-equivalent materials with desired radiological properties have been developed. Examples include the lung tissue substitute developed by Griffith et al. [16], a second lung tissue substitute LLLL1 developed at LLNL [17], and ALT2 developed by Traub et al. [18]. Although these developed tissue-equivalent materials exhibit promising radiological properties, the YM of PU-based foam is about 500 kPa, which is several orders of magnitude larger than typical values for human lungs and thus far from the desired range for human lung.

### BRIEF DESCRIPTION OF THE DRAWINGS

FIG. 1 [30]. Natural (red) and synthetic (blue) polymers used as raw material in bio-printing applications.

FIG. 2 [43]. Chemical structure of alginate blocks.

FIG. 3 [45]. Interaction mechanism of alginate and divalent cations.

FIG. 4 [52]. Young's modulus of alginate scaffolds: 3D-ALG (3.5 wt % alginate), 3D-ALG/PLA1 (3.5 wt % alginate+1% PLA) and 3D-ALG/PLA2 (2.5% alginate+2% PLA).

FIG. 5 [36]. Compression modulus of (a) pure alginate hydrogels at various concentrations, and (b) gelatin-alginate blend hydrogels at different concentrations up to 14 days at 37° C.

FIG. 6 [53]. Elastic modulus of partially cross-linked alginate hydrogel exposed to different CaCl₂) concentrations for 10 mins.

FIG. 7 [54]. Young's modulus for the high and low molecular weight alginate hydrogel crosslinked with different crosslink initiators.

FIG. 8 [62]. Simulation of mass attenuation coefficients of CaCO₃ and experiments in photon energy range of 1-1500 keV

FIG. 9 [62]. Simulation of mass attenuation coefficients of CaSO₄·2H₂O and experiments in photon energy range of 1-1500 keV

FIG. 10 [62]. Simulation of mass attenuation coefficients of BaSO₄ and experiments in photon energy range of 1-1500 keV

FIG. 11. Schematic of the synthesis procedures of alginate hydrogels.

FIG. 12. [113]. CO₂ pressure-temperature phase diagram.

FIG. 13. Viscosity of sodium alginate solutions with different concentrations under different testing speeds

FIG. 14. Exponential fitting of viscosity-concentration relation at 50 rpm.

FIG. 15. Exponential fitting of viscosity-concentration relation at 60 rpm.

FIG. 16. Exponential fitting of viscosity-concentration relation at 100 rpm.

FIG. 17. Intrinsic viscosity as a function of testing speed

FIG. 18. Morphologies of alginate hydrogels with different alginate concentrations (A) and different Ca²⁺:—COOH molar ratios (B) FIG. 19. SEM images of hydrogels with different alginate concentrations. (A) 1.5%, (B) 2%, (C) 2.5%, and (D) 3%.

FIG. 20. SEM images of hydrogels with different Ca²⁺:—COOH molar ratios. (A) 0.18, (B) 0.27, and (C) 0.36.

FIG. 21. Porosity of alginate hydrogels with different alginate concentrations.

FIG. 22. Porosity of alginate hydrogels with different Ca²⁺:—COOH molar ratios.

FIG. 23. FTIR spectra of alginate hydrogels with different alginate concentrations.

FIG. 24. FTIR spectra of alginate hydrogels with different Ca²⁺:—COOH molar ratios.

FIG. 25. Experimental setup for tensional testing FIG. 26. Tensional stress-strain behavior of alginate hydrogels with different alginate concentrations.

FIG. 27. Tensional tress-strain behavior of alginate hydrogels with different Ca²⁺:—COOH molar ratios.

FIG. 28. Tensional E0 as a function of alginate concentration.

FIG. 29. Tensional E₀ as a function of Ca²⁺:—COOH molar ratio FIG. 30. Experimental setup for compression testing.

FIG. 31. Compressive stress-strain behavior of alginate hydrogels with different alginate concentrations.

FIG. 32. Compressive stress-strain behavior of alginate hydrogels with different Ca²⁺:—COOH molar ratios.

FIG. 33. Exponential fitting of compressive stress-strain relation of alginate hydrogels with different concentrations.

FIG. 34. Exponential fitting of compressive stress-strain relation of alginate hydrogel with different Ca²⁺:—COOH molar ratios.

FIG. 35. Compressive E₀ as a function of alginate concentration.

FIG. 36. Compressive E0 as a function of Ca²⁺:—COOH molar ratio.

FIG. 37. Ultimate compressive stress and strain as a function of alginate concentration.

FIG. 38. Ultimate compressive stress and strain as a function of Ca²⁺:—COOH molar ratio.

FIG. 39. X-ray μ/ρ of alginate hydrogel with concentration of 5% and Ca²⁺:—COOH molar ratio of 0.18.

FIG. 40. Air volume ratio range of the hydrogel foam.

FIG. 41. Morphologies of alginate hydrogel foams with different air volume ratios

FIG. 42. Optical microscopy images of hydrogel foams with different air volume ratios (A) 50, (B) 100, and (C) 110.

FIG. 43. Number of pores and pore diameter distribution of alginate hydrogel foam sample 50.

FIG. 44. Number of pores and pore diameter distribution of alginate hydrogel foam sample 100.

FIG. 45. Number of pores and pore diameter distribution of alginate hydrogel foam sample 110.

FIG. 46. Compressive stress-strain behavior of alginate hydrogel foams with different air volume ratios.

FIG. 47. Exponential fitting of compressive stress-strain relation of alginate hydrogel foams for different air volume ratios.

FIG. 48. Compressive E0 of hydrogel foams with different air volume ratios.

FIG. 49. Ultimate compressive stress and strain of alginate hydrogel foams with different air volume ratios.

FIG. 50. CT image of alginate hydrogel foam (50).

FIG. 51. CT image of alginate hydrogel foam (100).

FIG. 52. CT image of alginate hydrogel foam (110).

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” or “approximately” means the recited value or plus or minus 20 percent of the recited value, so that, for example, “about 0.125” means 0.125±0.025, and “about 1.0” means 1.0±0.2.

As used herein, the term “radiation therapy” or radiotherapy refers to a cancer treatment that uses high-energy particles or waves, such as x-rays, gamma rays, electron beams, or protons, to destroy or damage cancer cells. Radiation therapy does not kill cancer cells right away. It takes days or weeks of treatment before DNA is damaged enough for cancer cells to die. Then, cancer cells keep dying for weeks or months after radiation therapy ends. External radiation (or external beam radiation) is the most common type of radiation therapy used for cancer treatment. A machine is used to aim high-energy rays or particles from outside the body at the tumor. The machine is large and may be noisy. It does not touch the patient's body, but can move around it, sending radiation to a part of the patient body from many directions. External beam radiation therapy is a local treatment, which means it treats a specific part of the body. For example, if the patient has cancer in the lung, the patient will have radiation only to the chest, not to the whole body.

Internal radiation therapy is a treatment in which a source of radiation is put inside the body. The radiation source can be solid or liquid. Internal radiation therapy with a solid source is called brachytherapy. In this type of treatment, seeds, ribbons, or capsules that contain a radiation source are placed in the body, in or near the tumor. Like external beam radiation therapy, brachytherapy is a local treatment and treats only a specific part of the body. (https://www.cancer.gov/about-cancer/treatment/types/radiation-therapy, https://www.cancer.org/cancer/managing-cancer/treatment-types/radiation/basics.html #:˜:text=xray)

As used herein, the term “dosimetry” or “radiation dosimetry” refers to the measurement, calculation and assessment of the ionizing radiation “dose absorbed” by an object, usually the human body, which is the amount of radiation energy that is deposited in tissue divided by the mass of the tissue. Ionizing radiation can be applied both internally, using ingested or inhaled radioactive substances, or externally using sources of radiation. Internal dosimetry can be assessed using a variety of monitoring, bio-assay or radiation imaging techniques, and external dosimetry can be determined using a dosimeter, or deduced from measurements made by other radiation detection instruments.

As used herein, the term “phantom” refers to a highly specialized device or model that simulates an animal (e.g. human) body, an animal organ, or a part of a body or organ. A phantom is utilized in medical imaging or radiation therapy for quality control, equipment calibration, dosimetry, and education. There are two main types of phantoms, anthropomorphic and calibration. A calibration phantom is often shaped as a cylinder or plate with densities of already known values, and it is utilized for quality control of imaging system. When reconstructing the imaged phantom, deviation from the correct density values can indicate a need for a new calibration of the imaging equipment. (radiopaedia.org/articles/phantom #:˜:text=Anthropomorphicphantoms). Anthropomorphic phantoms are made of materials having similar properties to human tissues, and they can be used to determine the optimal dose of radiation. Recent advancements in 3D printing technology can improve anthropomorphic phantoms to better mimic patient tissues. The anthropomorphic phantoms' basic design may involve having an outer plastic shell in appropriate anatomical shape that represents an animal body, an animal organ, or a part of a body or organ, or a section of an animal body. The shell can be filled with water and has a space that accommodates the imaging phantom insert or the dosimetry phantom insert. The dosimetry phantom insert can be designed to hold radiochromic film, and thermo-luminescent dosimeter (TLD), a passive radiation detection device, can be inserted into the phantom for the treatment. The imaging inserts have geometry and structures that can be seen with CT or MRI. (irochouston.mdanderson.org/rpc/services/Anthropomorphic_Phantoms/Phantoms.htm) Radiation therapy phantom is to simulate as closely as possible the human body in order to determine the amount of radiation being absorbed in various layers of the human body or to improve the accuracy and measurements of radiation dosimetry.

### 2. Overview

One objective of this disclosure is to provide materials with mechanical and radiological properties equivalent to human organs (e.g. lung), which is met by developing a hydrogel-based material that has mechanical and radiological tissue-equivalent properties to human lung.

The molecular weight of sodium alginate was characterized through measurement of the intrinsic viscosity and Mark-Houwink equation, which matched the specification from the manufacturer. Alginate hydrogels with different concentrations and Ca²⁺:—COOH molar ratios were synthesized by direct mixing of sodium alginate solution, CaCO₃ and GDL. Morphology analysis showed that homogeneous, transparent and three-dimensionally stable hydrogels were formed. There was no apparent difference in the physical appearance of the hydrogels. The three-dimensional stability of the hydrogels indicates that a strong network structure was formed within the hydrogels. The density of the hydrogels was characterized, exhibiting very close result to that of human lung tissue. SEM images of the microstructure of alginate hydrogels were characterized after CPD process. The images showed highly porous structures and uniformity of the hydrogels. The porosity of the alginate hydrogels was analyzed. The decrease in porosity of the hydrogel with increase of alginate concentration as well as Ca²⁺:—COOH molar ratio indicates the increase in the volume of the 3D connected structures.

Peak shifting was observed in the FTIR analysis for alginate hydrogels with different Ca²⁺: —COOH molar ratios, indicating that with an increase of Ca²⁺:—COOH molar ratio, stronger connections are formed within the hydrogel. Meanwhile the peak locations of alginate hydrogels with different concentrations were largely unchanged, indicating no obvious change of the molecular structure.

Both tensional and compressive stress-strain curves of the hydrogels were determined, and initial stage Young's modulus E₀ was calculated. For tensional testing, stress-strain curve behaved linearly before fracture exceeded 20% due to stress concentration at the edges of the anchoring grips, indicating a relatively large deformation could be achieved. Linear regression analysis was applied to the calculated E₀ and empirical relationships were developed between E₀ and concentration as well as Ca²⁺:—COOH molar ratio. For compressive testing, stress-strain relationship behaves nonlinearly on the entire stress-strain curve. Exponential fitting was applied, and the empirical relationship was developed. E₀ for compressive testing was also investigated within the initial 20% strain. Linear regression analysis was applied, and empirical relationships were established as well. By comparing the tensional and compressive E₀ results, it was found that the compressive E₀ were higher than that from tensional results. Due to the nature of compressive testing, the ultimate compressive stress and strain were also analyzed. Linear regression analysis was applied, and empirical relationships were developed. It was observed that the ultimate strain for all samples stabilized at 50%.

The radiological properties of the synthesized hydrogels were calculated, including elemental composition, ρₑ, Z_{eff} and I. They all demonstrated promising results compared with those of human lung.

The targeted air volume ratio range of hydrogel foam was calculated based on the HU range of human lung of 60%-70%, as guidance for the subsequent hydrogel foam preparation. Hydrogel foams with different air volume ratios were prepared by mixing of sodium alginate solution, CaCO₃, GDL and SLES 70 with mechanical mixing. Morphology analysis showed homogeneous, three-dimensionally stable (i.e., able to hold its three-dimensional shape) hydrogel foams were formed. There was no apparent difference in the physical appearance of the hydrogel foams. The free-standing of the hydrogel foams indicates that the network formed inside of the hydrogel is strong enough to support the foam itself. The density ρ of the hydrogel foams was calculated, giving close but lower densities compared with the calculated result. Optical microscopy images of the hydrogel foams were obtained, the number of pores and pore diameter distribution were analyzed. The results showed a clear image of the pores of different diameters. The pore diameter was approximately evenly distributed from tens of micrometers to about 500 μm. The diameter of pores slightly decreased with the decrease of volume of air. However, the number of pores demonstrated a trend of increasing within the area characterized.

Compressive testing was performed on the alginate hydrogel foams to analyze the mechanical properties, similar to the hydrogels. The stress-strain relationship of alginate hydrogel foams behaved nonlinearly on the entire stress-strain curve. Exponential fitting was also applied, and the empirical relationships were developed. The E₀ for compressive testing was then investigated within the initial 20% strain. The ultimate compressive stress and strain were also analyzed. The ultimate strain for all hydrogel foam samples was found to be stable at 38%.

The CT image of hydrogel foams were obtained and the HU values of hydrogel foams with different air volume ratios were measured. The CT images showed that mostly homogenous hydrogel foams were formed. The measured HU values compared favorably with results from theoretical calculations.

### 3. Embodiments of the Invention

### A. Exemplary Embodiments

According to one embodiment, provided is an alginate hydrogel that includes sodium alginate, CaCO₃, and D-(+)-gluconic acid δ-lactone (GDL), wherein, optionally, the GDL has a MW of about 178.

Also are provided different method of making an alginate hydrogel. For example, according to one embodiment, provided is a method of making an alginate hydrogel that involves:
i. dissolving sodium alginate in deionized water and continuously stirring the solution until homogeneous transparent to light yellow solution is obtained,
ii. placing the alginate solution in a vacuum chamber at approx. 85 kPa for approx. 24 hours to remove dissolved gas,
iii. admixing components into the alginate solution to form a mixture as follows:
   a. adding CaCO₃ to the alginate solution, and mixing the mixture for about 1 min, and
   b. adding GDL to the mixture and mixing the mixture for about 1 min, and
iv. sealing the mixture to gel for approx. 24 hours to form homogeneous hydrogels.

In specific examples, the alginate concentration (weight/volume) of the hydrogel is between about 0.5% and about 5%, optionally 1.5%, 2%, 2.5%, or 3%, and wherein Ca²⁺:—COOH molar ratio is between about 0.15 and about 0.4, optionally 0.18, 0.27, or 0.36. Also, in another example, the CaCO₃:GDL molar ratio may be approximately 0.5. In another example, the intrinsic viscosity of sodium alginate solution may be in the range of 0.88-1.06 μmL/g depending on the testing speed, and the molecular weight of sodium alginate is in the range of about 4.4×10⁴ to about 5.3×10⁴ g/mol. The alginate hydrogel may have a homogeneous, transparent, and three-dimensionally stable network structure and time stability over a duration of several weeks. In a specific example, the density of the alginate hydrogel is approximately 1 g/cm³. In another example, the alginate hydrogel may have a highly porous structure and uniformity, wherein the pore size ranges from tens to hundreds of nanometers, and wherein the porosity ranges from about 40% to about 50%.

In other examples, in FTIR spectra, the alginate hydrogel exhibits an absorption peak between 3600-2800 cm⁻¹ combined with an absorption peak at 2920 cm⁻¹, which are attributed to the stretching vibration of O—H and C—H, adsorption peaks at 1594 cm⁻¹ and 1406 cm⁻¹, which are attributed to asymmetric stretching vibration of COO⁻, and absorption peaks at 1081 cm⁻¹ and 1124 cm⁻¹, which are attributed to the C—O stretching vibration when the alginate concentration is between 0.5% and 5%, optionally 1.5%, 2%, 2.5%, or 3%, and the Ca²⁺:—COOH molar ratio is about 0.18, and wherein as Ca²⁺:—COOH molar ratio increases from about 0.18 or about 0.27 to about 0.36, and the peak location of —COOH shifts from 1591-1592 cm⁻¹, 1588-1589 cm⁻¹ to 1586-1587 cm⁻¹.

In addition, the initial stage Young's modulus (E₀) of tensional stress of the alginate hydrogel may range from about 2 kPa to about 25 kPa depending on the alginate concentration and the Ca2+:—COOH molar ratio. Also, the initial stage Young's modulus (E₀) of compressive stress of the hydrogel may range from about 4 kPa to about 35 kPa depending on the alginate concentration and the Ca2+:—COOH molar ratio. The ultimate compressive stress (Eᵤ) of the hydrogel may increase from about 18.15 kPa to about 75.21 kPa as the alginate concentration increases from about 1.5% to about 3%, while the ultimate strain of the hydrogel remains stable at about 50%, and wherein the ultimate compressive stress (Eᵤ) of the hydrogel increases from about 34.2 kPa to about 102.85 kPa as the Ca²⁺:—COOH molar ratio increases from about 0.18 to about 0.36, while the ultimate strain of the hydrogel remains at about 50%.

In further examples, the elemental compositions of H, O, Na and Ca in the hydrogel may be within 30% or 20% similarity to those of human lung, but wherein C is about 0.005 to about 0.0030 wt. percent. In another example, the electron density (ρₑ) of the hydrogel (3.40×10²³−3.50×10²³ g/cm³) may be within 30% or 20% similarity to that of a human lung, wherein the electron density is, optionally, about 3.35×10²³ g/cm³. In another example, the alginate hydrogel of claims 1-4, wherein effective atomic number (Z_{eff}) of the hydrogel (7.4-7.5) is within 30% or 20% similarity to that of a human lung, wherein the effective atomic number is, optionally, about 7.49. Wherein the mean excitation energy (I) of the alginate hydrogel (69 eV-70 eV) may be within 30 or 20% similarity with that of a human lung approx., wherein the mean excitation energy is about 75.2 eV According to other embodiments, provided is an alginate hydrogel foam made of sodium alginate, CaCO₃ (MW 100.09), D-(+)-gluconic acid δ-lactone (GDL), wherein, optionally, the GDL has a MW of approximately 178, and sodium lauryl ether sulfate. In a specific embodiment, the alginate hydrogel foam may be produced by a method comprising the steps of,
i. dissolving sodium alginate in deionized water to produce an alginate solution and continuously stirring the solution until homogeneous transparent to light yellow solution is obtained, and
ii. admixing components to form a mixture as follows:
   a. adding sodium lauryl ether sulfate into the alginate solution and stirring the mixture until fully mixed,
   b. adding CaCO₃ to the mixture and continuously stirring it until the mixture is fully mixed and the color of the mixture becomes white,
   c. adding GDL to the mixture to initiate gelation while mechanically mixing the mixture during the entire process, and
   d. stopping mechanical mixing when the volume of the mixture no longer changes or when the mixture fills the entire space of the container.

In a specific embodiment, the alginate hydrogel foam may have an alginate concentration (weight/volume) of between about 0.5% and about 5%, optionally 5%, and a Ca²⁺:—COOH molar ratio of between about 0.15 and about 0.4, optionally 0.18. In a specific example, the CaCO₃:GDL molar ratio is approximately 0.5. The concentration of sodium lauryl ether sulfate in the alginate hydrogel foam may be approximately 1.4% (weight/volume). The X-ray mass attenuation coefficients (μ/ρ) of the alginate hydrogel foam from theoretical calculation at alginate concentration of about 5% and Ca²⁺:—COOH molar ratio of about 0.18 may be from about 4.03×10³ cm²/g to about 1.81×10⁻² cm²/g when photon energy is from about 1×10⁻³ MeV to about 2×10 MeV. The air volume ratio of the hydrogel foam from theoretical calculation may stabilize at about 60 to about 70% when photon energy is from about 1×10⁻³ to about 2×10 MeV. Moreover, the alginate hydrogel foam may be homogeneous and three-dimensionally stable.

In a further example, the density ρ of the hydrogel foam may be from about 0.4 g/cm³ to about 0.8 g/cm³. In addition, the alginate hydrogel foam may have a pore diameter that is approximately evenly distributed from tens of micrometers to about 500 micrometers, and wherein the number of pores is about 103 to about 115 within an area of 5.7 mm².

In other examples, the compressive E₀ of the hydrogel foam may increase from about 10.86 kPa to about 18 kPa as the air volume ratio degreases from about 52.4% (Sample 50 to 39.0% (Sample 100), and wherein ultimate compressive stress (Eᵤ) may increase from 9.84 kPa to 17.58 kPa as the air volume ration decreases from 52.4% (Sample 50) to 32.4% (Sample 110), while the ultimate strains remain stable at approximately 38%. The alginate hydrogel foam may have a HU value that ranges from −290 to −510 as the air volume ration increases from 39.0% (Sample 100) to 52.4% (Sample 50).

According to another embodiment, provided is a phantom insert made of the alginate hydrogel foam useful for dosimetry, for example, and which can be implemented in an anthropomorphic phantom The anthropomorphic phantom may have an outer shell having an anatomical shape and an inner space that accommodates the phantom insert. In a further example, the shell can be filled with water, saline or artificial interstitial fluid. The size, dimension, and shape of the phantom insert is adapted depending on its intended use, such as being suitable for radiation therapy to a target organ or tissue, in particular human lung. In addition, the phantom insert can be designed to hold radiochromic film.

In other embodiments, provided is an organ phantom comprised of an alginate material described herein. In one example, the organ phantom is comprised of an alginate hydrogel foam.

### B. Materials

Hydrogels have been widely used in tissue-engineering. Their structures and properties are similar to extra cellular matrix (ECM), they have abundant source, and their properties are easy to control. In this disclosure, an alginate hydrogel material with controlled density ρ, elasticity and radiological property which is similar to human lung tissue was developed. This will provide a general solution to the manufacturing of tissue-like phantoms with tunable elasticity and radiological properties. The property of the material can be readily adjusted to suit any human tissue and organs.

### (1) Tissue-Substitute Materials

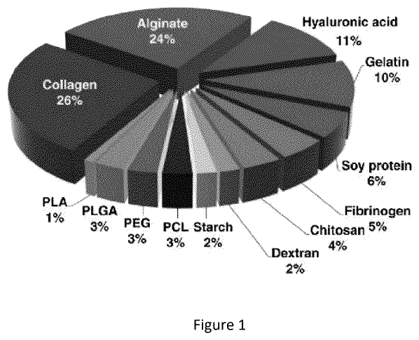

Choosing appropriate material is the key to production of phantoms with desired properties, as discussed above. Hydrogels are polymers in the form of 3D hydrophilic macromolecular networks. There are two types of hydrogels, natural and synthetic. Naturally derived hydrogels, including collagen, alginate, hyaluronic acid, gelatin, chitosan, are the most commonly used candidates as biomaterials, shown in FIG. 1, because of their similar properties to the ECM of natural tissues in many aspects, and their ability to be readily engineered.

### (a) Collagen

Collagen is the main structural protein in the mammal body. It has been widely used in tissue engineering applications due to its favorable biological compatibility [19]. Type I collagen is the main component of ECM and it has been widely used for wound healing and tissue regeneration [20]. However, there are several shortcomings for type I collagen which make it unfavorable for potential 3D phantom construction. The gelation rate of type I collagen is slow (about half an hour at 37° C.), and collagen will remain in a liquid form for more than 10 mins [21]. Another shortcoming is that it is liquid at low temperature and will form a fibrous structure when the temperature increases. Those properties limit the applications of collagen for future phantom development.

### (b) Hyaluronic Acid

Hyaluronic acid is an anionic, non-sulfated, glycosaminoglycan that widely exists in natural ECM. It has high biocompatibility and degradability, which makes it a promising material in bioengineering. However, the hyaluronic acid constructs are less stable because of their high hydrophilicity, which limits its applications [22].

### (c) Gelatin

Gelatin is a naturally-derived water-soluble protein from collagen through hydrolytic degradation [23]. It has been used as raw material in tissue engineering because of its unique properties such as biocompatibility and biodegradability. However, gelatin without any pretreatment will form a gel when temperature goes under 29° C., and will dissolve as a colloidal solution under 37° C. [24]. For this reason, gelatin is rarely considered a material used to produce phantoms, which requires thermal stability. Gelatin only comprises 10% of all polymers used in bio-printing.

### (d) Chitosan

Chitosan is a linear amino polysaccharide. It is the second most abundant naturally-derived polymer on earth after cellulose, which is mainly derived from chitin [25]. Chitosan is semi-crystalline in nature and is one of the main components of exoskeleton of crustaceans and insects. It has been widely used in wound healing, cartilage regeneration and other tissue engineering applications [26] because chitosan is the only positively charged natural polysaccharide, which makes it have great advantage in hemostasis and anti-bacterial applications [27]. However, chitosan is not soluble in aqueous solution when pH is above 7, and it becomes completely soluble when pH is less than 5 [28]. This limits its application unless some chemical modifications are applied.

### (e) Alginate

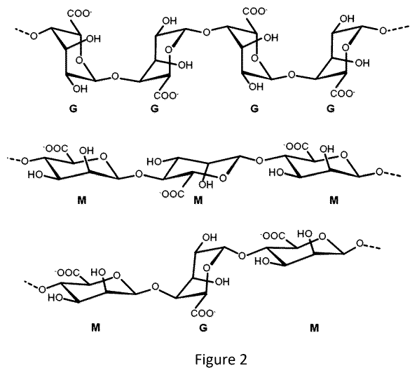

Alginate is an anionic polysaccharide existing widely in the cell walls of brown seaweed [41], which is very similar to ECM. The building blocks of alginate including sugar acid β-D-mannuronic acid (M) and its C-5 epimer, α-L-guluronic acid (G) link together to form linear molecular chain with (1,4) glycosidic bonds [29, 30]. The chemical structure of blocks of alginate is shown in FIG. 2.

It has been shown that M-blocks and G-blocks join together to form the linear polymeric chain in the form of homopolymeric M-blocks (MMMMMM), homopolymeric G-blocks (GGGGGG) and heteropolymeric M- and G-blocks (MGMGMG) [29, 30]. According to Gacesa et al. [32], the G-blocks region has higher stiffness than M-blocks due to the bonding difference between G-G, M-M and M-G. Therefore, the stiffness sequence from high to low will be G-block region, M-G block region and M-block region. It is noted that there's only one carboxyl group on every block, no matter M-block or G-block. Therefore, the amount of carboxyl group was used in this disclosure to quantitatively describe the relations with Ca^{2+.}

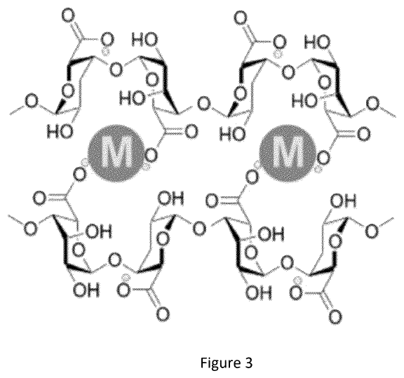

Alginate has advantages of quick gelling kinetics, mild reaction conditions, and environmental friendliness. It can easily and quickly form gel under favorably mild conditions in the presence of divalent cations, for example, Ca²⁺, Mg²⁺, Fe²⁺, Ba²⁺, or Sr²⁺[32], as shown in 10 FIG. 3. It is believed that the G-blocks mainly contribute to the formation of hydrogel because the structure of G-blocks allows a high degree of coordination of divalent cations [30, 31]. The binding of Ca²⁺ and G-blocks is believed to form an “egg-box” structure in which the Ca²⁺ forms the junctions between adjacent chains of alginate [34].

Usually, Ca²⁺ is one of the most used divalent cations to ionically cross-link alginate. The gelation speed is very fast in a mild aqueous condition, which makes it easy to control in the construction of phantoms.

Comparing the above five naturally derived polymers, alginate is the most desired material for use as tissue-substitute material compared with other materials. Alginate has similar properties to ECM and has an abundant source in nature. Besides, the initiating condition for gelling is only the contact of alginate molecule and divalent cations, the gelling speed is fast, which makes alginate hydrogel desirable for production of tissue-equivalent soft deformable phantoms.

### (2) Factors Influencing Material Property

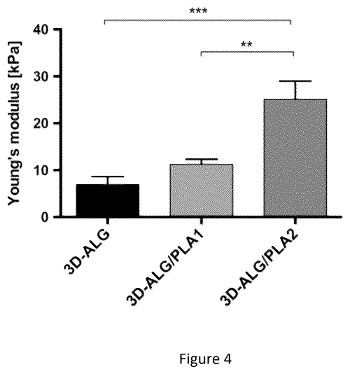

Although alginate is suitable for use as tissue-substitute material, its properties need to be carefully adjusted to meet the requirements mentioned above. The gelation speed can be controlled by molecular structure of alginate [35], temperature [36], release rate of divalent cations [37] and other conditions. The mechanical property of alginate is influenced by many factors, such as alginate type [29, 30, 32], concentration [38], gelling condition [35], molecular weight [33, 38], degree of polymerization, additives [39], and so on. Kosik-Koziol et al. [40] tested the Young's modulus of alginate hydrogel with PLA short sub-micron fiber reinforcement, as shown in FIG. 4:

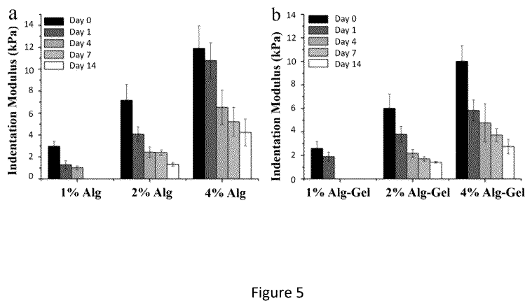

In the work of Chung et al. [24], the compression modulus of alginate and gelatin-alginate blends were tested, shown in FIG. 5:

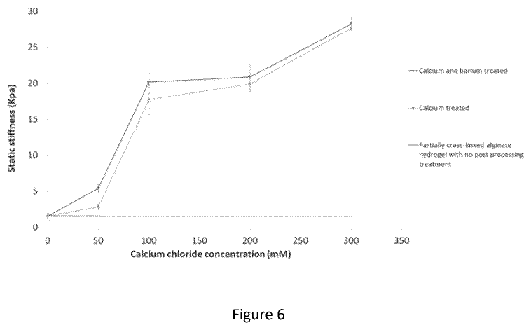

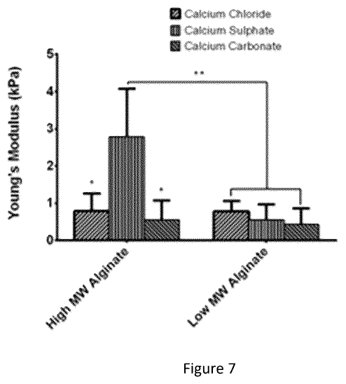

Tabriz et al. [41] investigated the influence of CaCl₂ concentration on the elastic behavior of alginate hydrogel. Freeman et al. [42] studied the influence of molecular weight and divalent cations on the Young's modulus of alginate hydrogel, as illustrated in FIGS. 6 and 7, respectively: From the above reported works, it can be concluded that the YM of alginate hydrogel is within the desired range for human lung and it can be tuned by adjusting different influencing factors. Therefore, alginate has a great potential as tissue-substitute material.

A review of previous studies indicates that most of the characterizations on the mechanical property of hydrogel were only performed under compression [37, 43-45]. Little has been reported on the tensile properties [35]. Considering the working mechanism of human lung, it is largely subjected to stretching conditions. Therefore, investigation of the tensile properties of the material is critical for future research and applications.

### (3) Radiological Properties

Tissue equivalent materials are materials with identical interaction behavior with given radiation type and energy as human tissue, which have a wide range of applications in diagnosis and therapeutic physics [46]. The search for tissue equivalent materials dated back to 1906 when Kienbock [47]proposed water as a kind of muscle equivalent material. From that time on, continuous efforts and progress have been made on the development of tissue-equivalent materials [48].

To measure the radiological properties of the material, a few parameters were selected to describe the interactions between the material and X-ray, including elemental composition, ρₑ, Z_{eff}, I, and μ/ρ, which are commonly used by those working in the related field. The related concepts and theory have been introduced above. The most important property is linear mass attenuation coefficient of the materials designed to be tissue equivalent. The materials which have identical linear attenuation coefficient as the targeted tissue are expected to have the same radiological performance. For design purposes, the linear attenuation coefficient is very sensitive to the elemental composition as well as ρ [49], which are difficult to control.

A few tissue-equivalent materials have been developed. Griffith et al. [16] developed a tissue-equivalent human-torso phantom made from PU with different concentrations of calcium carbonate (CaCO₃) to simulate the linear attenuation coefficients of different human organs, including human lung, named as Grif Taylor [17] introduced a second lung tissue equivalent phantom developed by Lawrence Livermore National Laboratory (LLNL), named LLLL1. Traub et al. [18] developed a novel lung tissue substitute using Foamex XRS-272, which contains 5.25% of CaCO₃ as property enhancement, named as ALT2. Tables 1 and 2 show the elemental composition, ρ, ρₑ, Z_{eff}, I and μ/ρ of human lung (ICRU-44) and the tissue-equivalent phantoms mentioned above.

TABLE 1
Radiological properties of human lung
tissue and tissue-equivalent materials

| | ICRU 44 | Grif[16] | LLLL1[17] | ALT2[18] |
|---|---|---|---|---|
| ρ | 0.26/1.06 | 0.26 | 0.28 | 0.26 |
| ρₑ | 3.35 × 10²³ | 3.28 × 10²³ | 3.31 × 10²³ | 3.29 × 10²³ |
| Z_{eff} | 7.49 | 7.49 | 6.97 | 7.37 |
| I | 75.2 | 70.9 | 68.1 | 70.2 |

TABLE 2
Mass Attenuation Coefficients of human lung
tissue and tissue-equivalent materials

| Energy (MeV) | ICRU 44 | Grif[16] | LLLL1[17] | ALT2[18] |
|---|---|---|---|---|
| 0.017 | 1.127E+00 | 1.113E+00 | 9.455E−01 | 1.108E+00 |
| 0.025 | 4.573E−01 | 4.612E−01 | 4.101E−01 | 4.600E−01 |
| 0.036 | 2.668E−01 | 2.731E−01 | 2.533E−01 | 2.689E−01 |
| 0.052 | 2.038E−01 | 2.046E−01 | 1.992E−01 | 2.035E−01 |
| 0.076 | 1.772E−01 | 1.754E−01 | 1.747E−01 | 1.754E−01 |
| 0.111 | 1.598E−01 | 1.571E−01 | 1.578E−01 | 1.575E−01 |
| 0.162 | 1.436E−01 | 1.409E−01 | 1.419E−01 | 1.414E−01 |
| 0.236 | 1.274E−01 | 1.248E−01 | 1.258E−01 | 1.253E−01 |
| 0.343 | 1.113E−01 | 1.091E−01 | 1.100E−01 | 1.095E−01 |
| 0.500 | 9.585E−02 | 9.388E−02 | 9.465E−02 | 9.423E−02 |

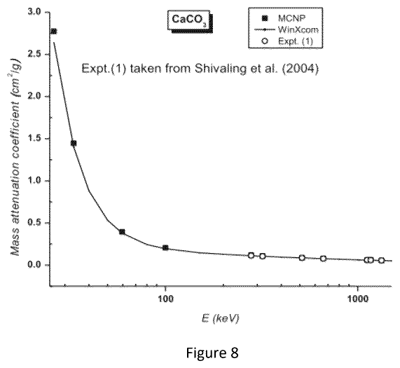

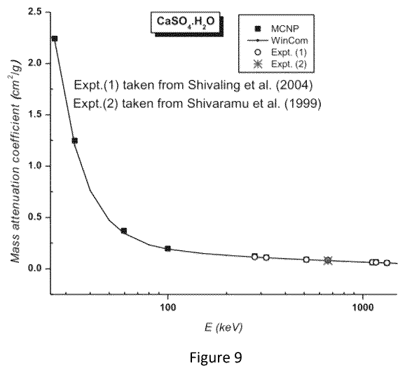

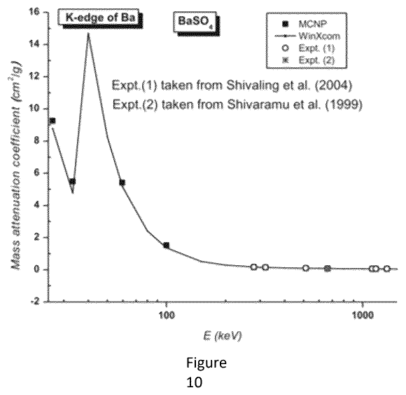

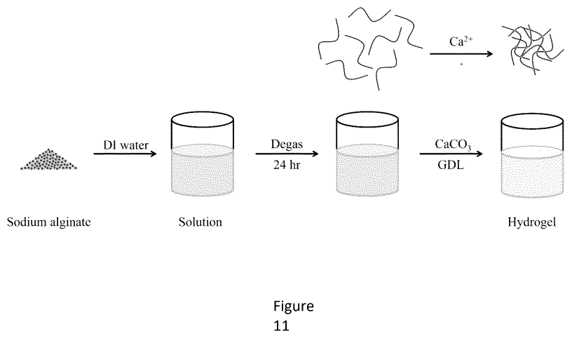

Application of dosimetric materials in medical and radiological protection is very important for exposure monitoring and protection. Several types of adjuvants can be used to adjust radiological properties of the material prepared to make it closer to the properties of human tissue or organs, such as AlOH, Al₂O₃, SiO₂, CaSO₄·2H₂O, CaCO₃, BaSO₄ [50, 51]. They are widely used to study the radiation interaction properties of actual human organs and tissues. Theoretical values for the μ/ρ of all elements and some compounds over wide photon energy range have been tabulated [52]. Using such a table the value for any compound can be obtained. El-Khayatt et al. [50] simulated the radiological property for multiple tissue-substitute materials, shown in FIGS. 8-10.

The above review has indicated that it is possible to apply those adjuvants in embodiments to obtain the desired radiological properties by carefully choosing the type and amount of the adjuvants.

Although ideal radiological properties have been achieved, almost all the phantom materials were composed of foaming plastics, which means the mechanical properties of the materials above cannot approach that of human lung. The YM of PU foam is about 500 kPa, which is far from desirable compared to that of human lung. Thus, it is necessary to develop a new material to meet both the mechanical and radiological property requirements of the real human lung.

### C. Phantoms for Dosimetry of Radiation Therapy

For radiation therapy, the treatment “absorbed dose” and any collateral “absorbed dose” need to be assessed and monitored. To this end, another objective of this disclosure to provide a phantom for human tissues and organs, in particular the lung. Here, alginate hydrogel made of sodium alginate, Ca²⁺, and GDL, and alginate hydrogel foam made of sodium alginate, Ca²⁺, GDL, and sodium lauryl ether sulfate, which have mechanical and radiological properties similar to human lung, are provided as materials to imitate human lung tissue for radiation therapy. The advantage of these materials, in addition to their being biodegradable and environment-friendly, is that those properties can be varied by adjusting the concentration of sodium alginate and the ratio of alginate and calcium. In addition, the size, dimension, and shape of the organ phantom are decided according to a radiation therapy target organ or tissue

Further, the concentration of sodium lauryl ether sulfate, which is used here as a surfactant, can be adjusted to increase or decrease the number and size of pores in the hydrogel foam, imitating certain organs having pores, such as lung and kidney. Thus, in one embodiment, a lung phantom imitating a lung lobe, which is made of alginate hydrogel foam, is provided. In another embodiment, a phantom kidney made of alginate hydrogel foam and containing pores whose diameters are similar to those of tubular lumens in the kidney can be provided.

In some embodiments, the alginate hydrogel phantom can be a liver-, pancreas-, gall bladder-, skeletal muscle- or brain phantom. In some embodiments, the alginate hydrogel phantom can be a phantom of organs having tubular structures such as small- and large intestines. In some embodiments, the alginate hydrogel phantom can be a phantom of an organ having a sac such as stomach, bladder and uterus, and a phantom of the heart.

In some embodiments, one hydrogel- or hydrogel foam phantom is put in a plastic shell having an appropriate anatomical shape to be used for radiation therapy dosimetry. The shell has a compartment that accommodates the phantom as an insert, and it is filled with water, saline or artificial interstitial fluid. In some embodiments, more than one hydrogel- and/or hydrogel foam phantoms are put in such a plastic shell to imitate complicated organ arrangement in the thoracic-, abdominal- or pelvic cavity for the improvement of dosimetry accuracy in radiation therapy. In addition, the organ phantom insert can be designed to hold radiochromic film.

### EXAMPLES

### Example 1. METHODOLOGY

### 1. Hydrogel Preparation and Characterization

### 1.1. Materials and Synthesis

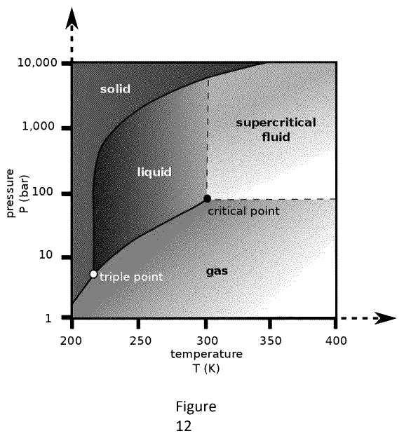

The alginate hydrogel was prepared using sodium alginate (product number: W201502), CaCO₃ (product number: 795445, molecular weight: 100.09) and D-(+)-gluconic acid δ-lactone (GDL) (product number: G4750, molecular weight: 178.14) from MilliporeSigma. Deionized (DI) water was used in the entire hydrogel preparation process. Sodium alginate was dissolved in DI water at room temperature with continuous stirring (600 rpm) until homogeneous transparent to light yellow solution was obtained. Then, the solution was placed in a vacuum chamber at 85 kPa for 24 hours to remove dissolved gas. CaCO₃ in combination with GDL was used as a source of Ca²⁺ to initiate gelation. The CaCO₃ to GDL molar ratio of 0.5 was used to maintain neutral pH value [37]. In one set of experiments, alginate hydrogels of different alginate concentrations (1.5%, 2%, 2.5%, 3%) were prepared with the Ca²⁺:—COOH molar ratio maintained at 0.18. In another set of experiments, the Ca²⁺:—COOH molar ratio was varied (0.18, 0.27, 0.36) while the alginate concentration was maintained at 2%. CaCO₃ suspension was added to each sodium alginate solutions and allowed to mix for 1 min. Then GDL solution was added to the suspension and mixed for another 1 min to initiate gelation. The schematic of the synthesis procedure of alginate hydrogels is illustrated in FIG. 11. The mixtures were sealed in petri dishes to gel for 24 hours to form homogeneous hydrogels. The alginate concentrations presented above and in the following sections represent the final mass/volume concentrations.

### 1.2. Intrinsic Viscosity and Molecular Weight Characterization

Intrinsic viscosity measures the contribution of the solute to the viscosity of the solution and it is related to the molecular weight of the solute. The intrinsic viscosity [η] is defined as:

$$[ η ] = \text{?} \frac{η - {η}_{0}}{{η}_{0} Φ} \qquad ( 7 )$$

$$\text{?} \text{indicates text missing or illegible when filed}$$

where η is the viscosity of the solution, η₀ is the viscosity of the solvent and Φ is the volume fraction of the solute in the solution. After sodium alginate solution was prepared, [η] of the solutions was calculated through the measured viscosities to obtain the molecular weight information of sodium alginate by applying the Mark-Houwink equation [53]:

$$[ η ] = {KM}^{a} \qquad ( 8 )$$

which gives a relation between the intrinsic viscosity and molecular weight of a polymer-solvent system where M is the molecular weight of the polymer. The value of the parameters K and a in Mark-Houwink equation depend on the particular polymer-solvent system, here in this specific alginate-water system, K=2.0×10⁻⁵ and a=1 [29]. Smidsrod et al. [54] demonstrated that the [η] of alginate at infinite ionic strength was representative of the uncharged alginate molecule, being identical to that obtained for the alginic acid formed in 0.1 M NaCl, when the macromolecule contains no charges. The sodium alginate solution used for viscosity testing is prepared by dissolving sodium alginate in 0.1 M NaCl solution. The viscosity of sodium alginate solution under different concentrations was tested using a Brookfield DVERV viscometer.

### 1.3. Density Characterization

In order to compare the p of prepared alginate hydrogels and that of human lung tissue, p of the alginate hydrogels were calculated by the relation:

$$ρ = m / V \qquad ( 9 )$$

through measuring the mass m and volume V of the hydrogel samples.

### 1.4. Critical Point Drying and SEM Characterization

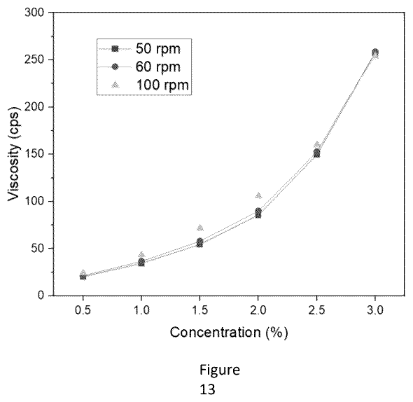

Scanning electron microscopy (SEM) characterization was performed on a Zeiss ULTRA-55 FEG SEM after the samples were critical point dried using an Electron Microscopy Sciences K850 Critical Point Dryer. It should be noted that a large portion of the previous studies used freeze-drying technique to remove water from the hydrogel. However, a reasonable concern regarding freeze-drying principle is that the formation, expansion and growth of ice crystals during freeze drying may cause damage to the microstructures of the samples, especially for fine structures, such as biological specimens or the 3D interconnected structure of hydrogel. This concern has been validated by relevant studies [55-58]. Therefore, critical point drying (CPD) was chosen as a more favorable way to remove water from the hydrogel. This technique uses CO₂ to replace water in the samples, which has a favorable critical point of 304.128K/30.978° C. and 7.3773 MPa, see FIG. 12 [59], compared to other transitional mediums. When temperature and pressure increase above the critical point, the surface tension between the gas and liquid states of CO₂ decreases to zero, thereby avoiding the damage of surface tension to the microstructures of samples during regular transitional stage. However, CO₂ is not miscible with water, therefore an exchange fluid which is miscible with both water and CO2 must be applied. Here isopropyl alcohol was used as the exchange fluid. The dehydration process was performed under a series of isopropyl alcohol solutions with increasing volume percentages of 10%, 30%, 50%, 70%, 90%, and 100% for 15 mins for every concentration. The magnification of the SEM images was maintained at 20K for all samples. ImageJ was used to calculate the porosity of the samples from the images obtained. The threshold to convert images from grayscale to binary was set as “Auto”. Averaged results and standard deviations were calculated for the porosity of the samples.

### 1.5. FTIR Characterization

Fourier transform infrared (FTIR) analysis was performed on a SHIMADZU IRSpirit instrument. First the FTIR spectra of the chemicals used in the hydrogel synthesis were characterized. Then the FTIR spectra of the synthesized alginate hydrogels were also characterized. Since over 95% composition of the hydrogels is water, the absorption peak of water must be very strong compared to other components, which has been verified in the pre-characterization experiments. Therefore, the hydrogel samples were first vacuumed at 40° C. for 48 hours to remove water completely. Then the samples were grounded into powder to in readiness for the analysis. The FTIR spectra were obtained in the range of 4000-600 cm⁻¹ and averaged over 40 scans at a resolution of 1.42 cm⁻¹. Each reported wavenumber value was averaged from three specimens.

### 1.6. Characterization on Mechanical Properties

Uniaxial tensional and compressive tests were performed to characterize the mechanical properties of alginate hydrogels on an MTS Criterion platform model 43 (MTS Systems Corporation, Eden Priarie, MN) and TESTRESOURCES 100 Series, respectively. The samples were cut into rectangular blocks and the dimensions of the blocks were measured to calculate the stress and strain. The crosshead speed was set at 5 mm/min for tensional testing and 10 mm/min for compressive testing. Three specimens were tested for each sample and the average and standard deviations were calculated from the results. Both tensional and compressive stress-strain curves of alginate hydrogels with different concentrations as well as Ca²⁺:—COOH molar ratios were obtained and the initial stage Young's modulus (E₀) was calculated. Empirical relationships of E₀ and alginate concentration as well as Ca²⁺:—COOH molar ratio were developed by applying linear regression analysis. The p-values were also calculated. For compressive test, the ultimate compressive stress and strain of the hydrogel materials were analyzed.

### 1.7. Theoretical Calculation of Radiological Properties

Radiological properties of the alginate hydrogels as human lung substitute were theoretically calculated following the method described above and compared with that of human lung tissue. The parameters considered include elemental composition, ρₑ, Z_{eff}, and I.

### 2. Hydrogel Foam Preparation and Characterization

### 2.1. Theoretical Prediction of Air Volume Ratio

The targeted air volume ratio of hydrogel foam is calculated based on the HU range of human lung, which is −600 to −700. The foam is assumed to be a simple mixture of air and synthesized alginate hydrogel. By applying simple additive rule of μ/ρ of the single elements, the fraction of air and synthesized alginate hydrogel can be obtained.

### 2.2. Materials and Synthesis

The alginate hydrogel foam was prepared using sodium alginate (product number: W201502), CaCO₃ (product number: 795445, molecular weight: 100.09) and GDL (product number: G4750, molecular weight: 178.14) from MilliporeSigma. Sodium lauryl ether sulfate 70% (SLES 70) from Renowned Trading LLC. DI water was used in the entire foam preparation process. Sodium alginate was dissolved in DI water at room temperature with continuous stirring (600 rpm) until homogeneous transparent to light yellow solution was obtained. CaCO₃ in combination with GDL was used as a source of Ca²⁺ to initiate gelation. SLES 70 was used as surfactant to create porous structure within the hydrogel. Alginate concentration was maintained at 5% and Ca²⁺:—COOH molar ratio was maintained as 0.18. The CaCO₃ to GDL molar ratio of 0.5 was also maintained during the foam preparation to maintain neutral pH value [37]. A 2% volumetric ratio of SLES 70 was added for all samples. Here were used beakers with lids to control the solution/air mixing ratio. The total volume of the beaker was 140 mL. Based on the theoretical calculation result, different volumes of the mixture (50 mL, 100 mL, 110 mL) were added into the beaker for mixing with air. The sample with 50 mL mixture was designed for mixing with sufficient air, the air volume ratio of the other two samples were controlled. SLES 70 was first added into the alginate solution and stirred with an electrical mixer until fully mixed. Then CaCO₃ suspension was added into the mixture and continuously stirred until fully mixed when the color of the mixture became white. Finally, the GDL solution was added into the mixture to initiate gelling with mechanical mixing maintained during the entire process. For the sample with 50 mL mixture, mechanical mixing was stopped when the volume of the mixture no longer changes. For samples with 100 mL and 110 mL mixture, mechanical mixing stopped when the mixture filled the entire space of the beaker.

### 2.3. Density Characterization

Similar to ρ characterization of the alginate hydrogels, ρ of the hydrogel foams were calculated by the definition of density using the relation in previous Equation (9) through measuring the mass m and volume V of the hydrogel foam samples.

### 2.4. Optical Microscopy

The optical micro images of the hydrogel foams were obtained on a Zeiss Axio Observer A1. ImageJ was used to calculate the number and diameter distribution of pores in the samples within a certain area from the images obtained. The shape of the pores was assumed to be circular.

### 2.5. Characterization on Mechanical Properties

Uniaxial compressive tests were performed to characterize the mechanical properties of alginate hydrogel foams on TESTRESOURCES 100 Series. The samples were cut into rectangular blocks and the dimensions of the blocks were measured to calculate the stress and strain. The 36 crosshead speed was set at 10 mm/min. Three specimens were tested for each sample and the average and standard deviations were calculated from the results.

### 2.6. Characterization on Radiological Properties

The prepared hydrogel foams were scanned on a Siemens SOMATOM Drive Dual Source CT Scanner. The scanning parameters were set as 40 mAs, 120 kVp, and 1 μmm slices. The DICOM files obtained were analyzed by MicroDicom DICOM Viewer. The HU values of five cross-sections were analyzed for each sample. The HU values obtained from CT scanning were compared with theoretical calculation results based on the density data obtained.

### Example 2. FINDINGS

### 1. Alginate Hydrogels

### 1.1. Intrinsic Viscosity and Molecular Weight

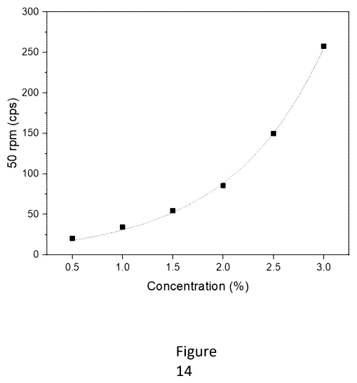

The viscosities of sodium alginate solution with different concentrations and testing speeds were obtained as shown in FIG. 13. Under the same testing speed, the viscosity of sodium alginate solution increases with the increase of concentration. For the solution at a certain concentration, a slight increase of the viscosity is observed when the rotation speed increases, which indicates the sodium alginate solution has the behavior of shear thickening fluid.

Exponential fitting was performed on the concentration-viscosity relation curves under different testing speeds. Here a two-parameter exponential function (Exp2P) was chosen as the fitting model and the results are shown as follows:

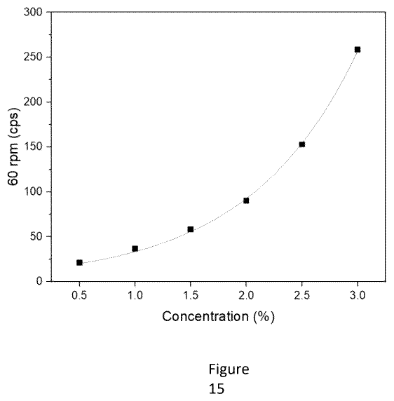

The fitting result at a testing speed of 50 rpm is shown in FIG. 14 and the corresponding fitting equation is:

$$y = 10.78 \times 2.8 {8}^{x} \qquad ( 10 )$$

Substituting Equation (10) into Equation (7), the intrinsic viscosity of sodium alginate solution at 50 rpm is found to be 1.06 mL/g.

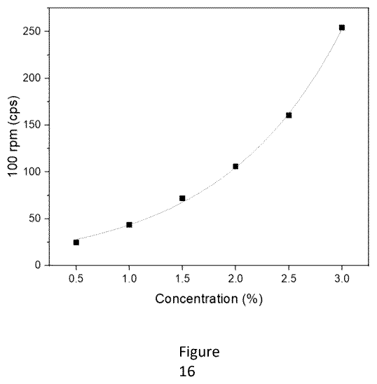

The fitting result at a testing speed of 60 rpm is shown in FIG. 15 and the corresponding fitting equation is:

$$y = 12.15 \times 2.7 {7}^{x} \qquad ( 11 )$$

Substituting Equation (11) into Equation (7), the intrinsic viscosity of sodium alginate solution at 60 rpm is found to be 1.02 mL/g.

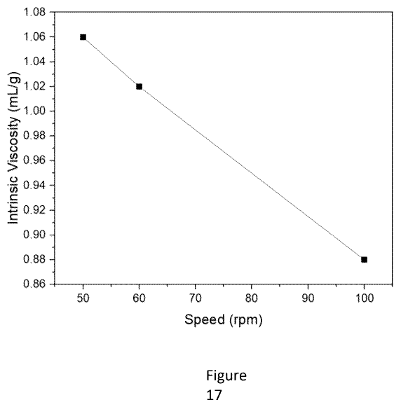

The fitting result at a testing speed of 100 rpm is shown in FIG. 16 and the corresponding fitting equation is:

$$y = 18.17 \times 2.4 {1}^{x} \qquad ( 12 )$$

Substituting Equation (12) into Equation (7), the intrinsic viscosity of sodium alginate solution at 100 rpm is found to be 0.88 mL/g.

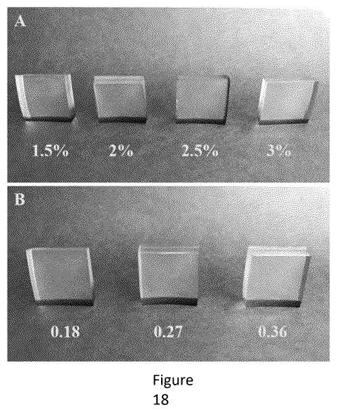

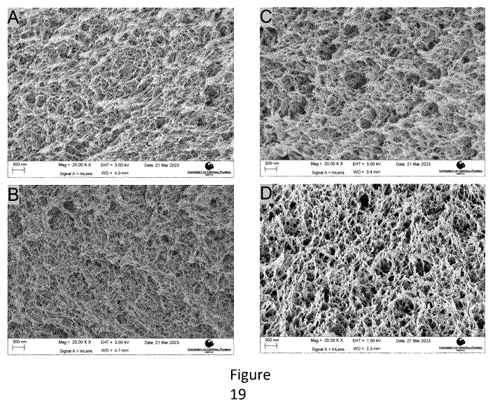

Considering the difference in the intrinsic viscosity of the solution due to the difference of testing speed, the relation between intrinsic viscosity and testing speed was also investigated as shown in FIG. 17. The intrinsic viscosity-speed relation is observed to be linear and the linear fitting equation is:

$$y = - 0.0036 x + 1.24 \qquad ( 13 )$$

It can be concluded that when the testing speed approaches 0 rpm, the intrinsic viscosity of sodium alginate solution approaches 1.24 mL/g. This value is independent of concentration as well as testing speed and can thus be considered as one of the inherent properties of sodium alginate solution.

Substituting the intrinsic viscosity values obtained from the experiment data into Equation (8), the molecular weight of sodium alginate can be obtained to be in the range of 4.4×10⁴-5.3×10⁴ g/mol, which is in the range of the data provided by the product manufacturer (1.2×10⁴−4.4×10⁴ g/mol). If the intrinsic viscosity value obtained from the linear fitting model when the speed is 0 rpm is substituted into Equation (8), the molecular weight of sodium alginate is obtained to be 6.2×10⁴ g/mol.

### 1.2. Morphology

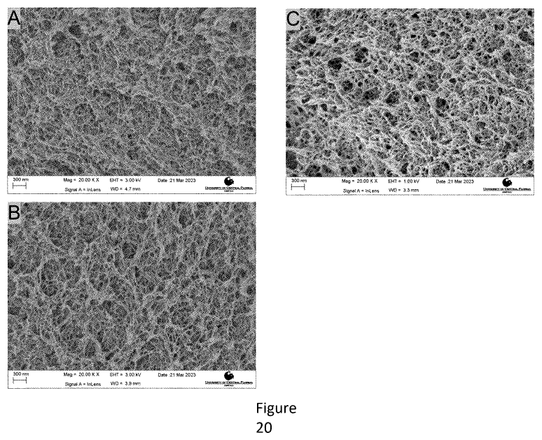

FIG. 18 shows the samples of alginate hydrogel material produced. All samples were cut into 20 mm×20 mm rectangular shapes with uniform thickness of 6 mm. The morphology of prepared samples indicates that homogeneous, transparent, and three-dimensionally stable alginate hydrogels were formed. There is no apparent difference in the physical appearance of the hydrogels irrespective of the alginate concentration and the Ca²⁺:—COOH molar ratio. The three-dimensional stability of the hydrogels indicates that a strong network structure was formed within the hydrogels. The hydrogels also demonstrate exceptional time stability over a duration of several weeks. In addition, the morphology remains intact except for a slight size shrinkage. The potential shrinkage of the hydrogels is attributed to the loss of water. Studies have been conducted on the swelling behavior of hydrogels [61, 62] and the volume of a hydrogel had direct connection with its water content. In this study, the initial amount of water was strictly controlled. Therefore, subsequent evaporation of water over time may cause shrinkage of the hydrogel.

### 1.3. Density

ρ of the hydrogels were calculated by measuring the mass and volume of the hydrogel samples and the results are presented in Table 3. The results show that the densities of all samples are slightly above 1 g/cm³, which is very close to that of human lung tissue that is 1.06 g/cm³.

TABLE 3
Density of alginate hydrogels

| Samples | 1.5%-0.18 | 2%-0.18 | 2.5%-0.18 | 3%-0.18 | 2%-0.27 | 2%-0.36 |
|---|---|---|---|---|---|---|
| Density | 1.052 | 1.012 | 1.028 | 1.013 | 1.008 | 1.008 |
| (g/cm³) | | | | | | |

### 1.4. SEM

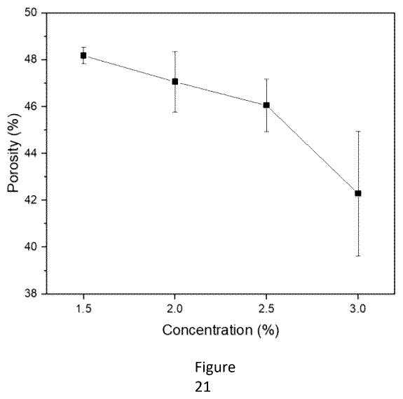

The SEM images of samples with different alginate concentrations are shown in FIG. 19. The corresponding SEM images for samples with different Ca²⁺:—COOH molar ratios are presented in FIG. 20.

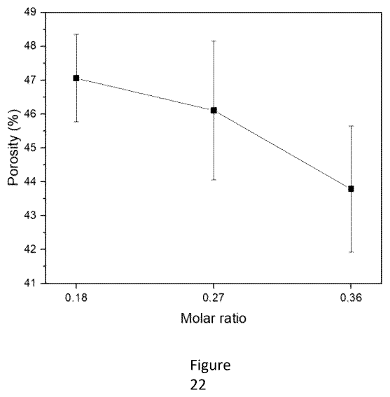

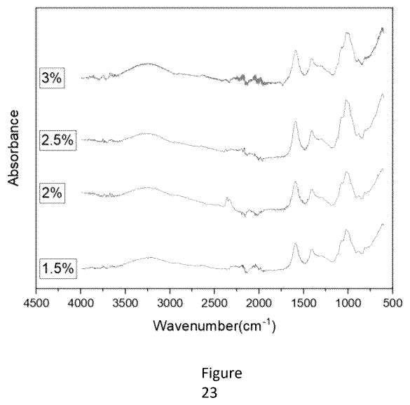

FIGS. 19 and 20 show that all samples exhibit highly porous structures and uniformity within the range of concentrations and Ca²⁺:—COOH molar ratios considered. The pore size of all samples ranges from tens to hundreds of nanometers. The porosity analysis corresponding to FIG. 19 and FIG. 20 are shown in FIG. 21 and FIG. 22, respectively.

FIG. 21 shows that the porosity decreases from 48.18% to 42.29% over the range of alginate concentration considered (1.5% to 3%). FIG. 22 shows the porosity decreases from 47.06% to 43.79% when the Ca²⁺:—COOH molar ratio increases from 0.18 to 0.36. The decrease in porosity of the hydrogel with increase of alginate concentration as well as Ca²⁺:—COOH molar ratio indicates the decrease in the volume of the pores and the increase in the volume of the 3D connected structures.

### 1.5. FTIR

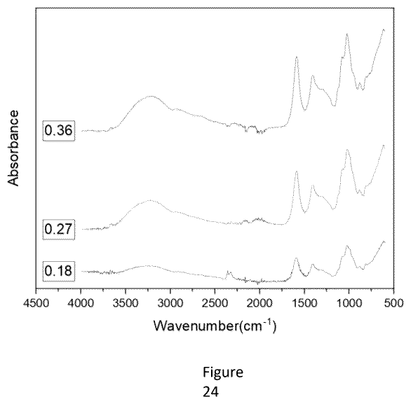

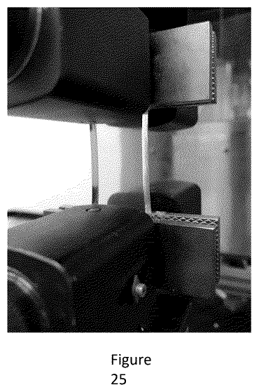

The FTIR spectra of alginate hydrogels with different concentrations as well as Ca²⁺:—COOH molar ratios are shown in FIGS. 23 and 24. Multiple peaks are observed in each spectrum. The broad absorption peak between 3600-2800 cm⁻¹ combined with a small broad peak at 2920 cm⁻¹ are associated with the stretching vibration of O—H and C—H. The strong-sharp peaks at 1594 cm⁻¹ and 1406 cm⁻¹ are the characteristic absorption peaks of asymmetric stretching vibration of COO—. Meanwhile, the weak-sharp absorption peaks at 1081 cm⁻¹ and 1124 cm⁻¹ are attributed to the C—O stretching vibration. In comparison with pure sodium alginate, the FTIR spectrum of alginate hydrogel exhibits a similar pattern but with shifted peak locations. For FTIR spectra of alginate hydrogels of different concentrations, the COO— peak locations as well as other peak locations are largely unchanged, which implies that the molecular structure is the same within these samples. This result is understandable because the only changing condition is the concentration of sodium alginate. After the vacuuming step in the preparation of the samples, nearly 100% of the water has been removed from the sample. Therefore, it can be concluded that the difference in the mechanical property of the alginate hydrogel, see in Section 1.6, is largely due to the density of 3D network within the hydrogel, which in turn is determined by the concentration of sodium alginate. Peak shifting occurs for FTIR spectra of alginate hydrogels with different Ca²⁺:—COOH molar ratios. As Ca²⁺:—COOH molar ratio increases from 0.18, 0.27 to 0.36, the peak location of —COOH shifts from 1591-1592 cm⁻¹, 1588-1589 cm⁻¹ to 1586-1587 cm⁻¹. This result indicates that with an increase of Ca²⁺:—COOH molar ratio, stronger interactions are formed within the hydrogel, which will increase the YM of the material.

### 1.6. Mechanical Properties

For the mechanical properties, tensional testing was conducted on the hydrogels in order to mimic the working condition of human lung. Compressive testing was also conducted on the hydrogels to compare the results with that obtained from tensional testing.

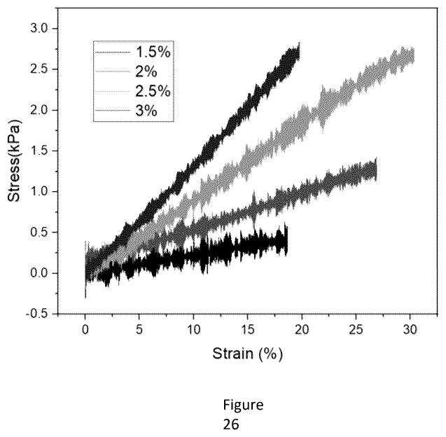

FIG. 25 shows the experimental setup of the hydrogel for tensional testing. The hydrogel samples were anchored in the MTS system parallel to the stretching direction. The signals were reset to zero to initiate the test. All samples were stretched until cracking occurred at the anchored ends due to local stress concentration.

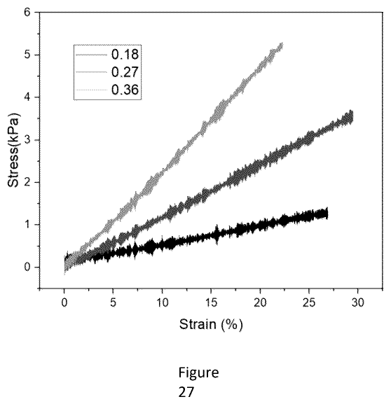

FIGS. 26 and 27 show the stress-strain results obtained for the samples investigated under tensional tests. FIG. 26 shows effects of different alginate concentrations while FIG. 27 shows effects of variation of Ca²⁺:—COOH molar ratios. The results indicate that all the samples have similar stress-strain behaviors, being approximately linear. The average strain obtained before fracture exceeds 20%, which indicates that a relatively large deformation can be achieved. The fracture pattern indicates that the failure of the tested samples is due to the stress concentration at the edges of the samples at the anchoring grips. Thus, it can be reasonably predicted that the elongation behavior of the material is no worse than the results obtained from the tested samples. Therefore, the YM obtained from the testing in this study is hereinafter referred to as the “initial stage Young's modulus” and denoted E₀.

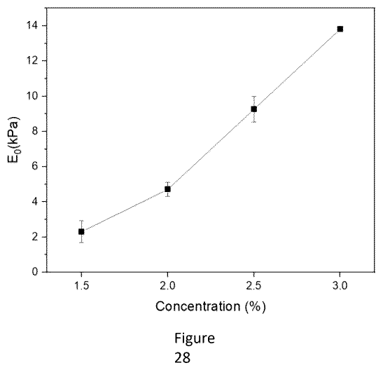

FIG. 28 shows the tensional E₀ as a function of alginate concentration. Every data point in the figure represents the average of three test results. The error bar represents the standard deviation of the data. The result shows that E₀ of the hydrogel increases linearly with the alginate concentration. E₀ increases from 2.01 kPa to 13.81 kPa when the alginate concentration increases from 1.5% to 3%. The following empirical relationship is obtained between tensional E₀ and concentration using linear regression analysis:

$$\text{?} = \text{?} x - \text{?} \qquad ( 14 )$$

$$where$$

$$\text{?} = 799 ( \frac{kPa}{g / mL} )$$

$$and$$

$$\text{?} = 10.53 kPa .$$

$$\text{?} \text{indicates text missing or illegible when filed}$$

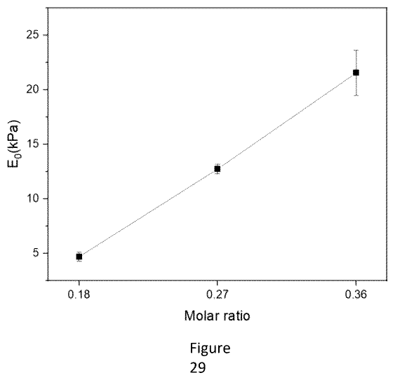

FIG. 29 shows E₀ as a function of Ca²⁺:—COOH molar ratio. Every data point in the figure represents the average of three test results. The standard deviation of the results is also shown in the figure as error bar. The functional relationship is observed to be linear as well, with E₀ increasing from 4.71 kPa to 21.57 kPa as Ca²⁺:—COOH molar ratio increases from 0.18 to 0.36. Linear regression analysis was applied, indicating the following relationship:

$${E}_{0}^{T} = {D}^{T} x - {F}^{T} \qquad ( 15 )$$

where D^{T}=93.67 kPa and F^{T}=12.28 kPa.

In addition, analysis of variance (with p-values) for E₀ as a function of both the alginate concentration and the Ca²⁺:—COOH molar ratio was performed to evaluate the statistical significance of the test results. The p-value obtained for E₀ as a function of alginate concentration was 0.006<0.05 and the p-value for E₀ as a function of Ca²⁺:—COOH molar ratio was 0.017<0.05.

FIG. 30 shows the experimental setup of the hydrogel sample for compressive testing. The hydrogel samples were placed between two parallel surfaces in the TESTRESOURCES 100 Series system perpendicular to the compression direction. The signals were reset to zero to initiate the test. All samples were compressed until cracking occurred.

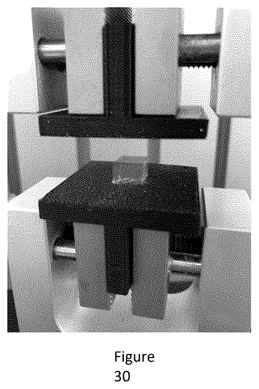

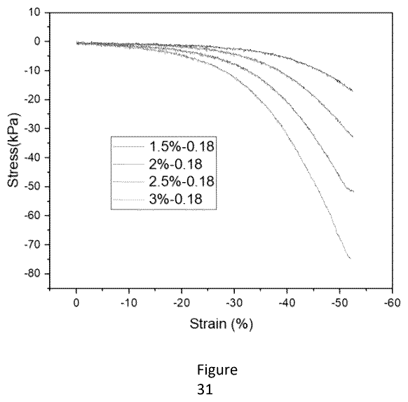

FIGS. 31 and 32 show the stress-strain relations of the prepared alginate hydrogels obtained from the compressive test. FIG. 31 presents the results as a function of alginate concentration while FIG. 32 shows the corresponding results for varying Ca²⁺:—COOH molar ratios. The results indicate that all the samples have similar non-linear stress-strain behaviors. For every individual sample, the stress increases faster with the increase of the strain. In FIG. 31, the stress increases with the increase of alginate concentration. In FIG. 32, the stress in each sample also increases with the increase of Ca²⁺:—COOH molar ratio.

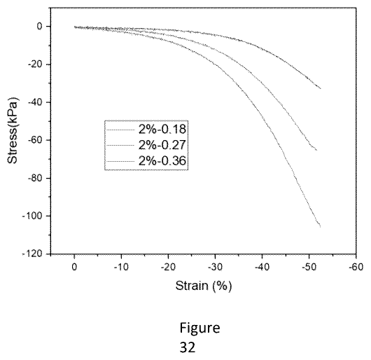

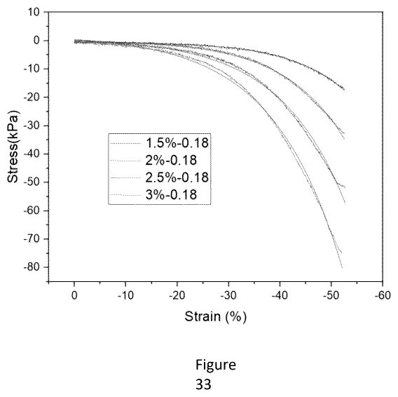

Similar to the viscosity-concentration relation analysis of the sodium alginate solution, exponential fitting was conducted for the compressive stress-strain relation of the hydrogels. Here function Exponential was chosen as the fitting model and the results are shown in FIG. 33 for the variation of alginate concentrations and FIG. 34 for the variation of Ca²:—COOH molar ratios.

The fitting equations for alginate hydrogels with different concentrations are as follows: When the alginate concentration is 1.5%, the fitting equation is:

$$y = {y}_{1.5}^{0} + {A}_{1.5} {e}^{{R}_{1.5} x} \qquad ( 16 )$$

where y_{1.5}⁰=−0.412 kPa, A_{1.5}=−0.123 kPa, and R_{1.5}=−0.0939.

The fitting equation for alginate concentration of 2% is:

$$y = {y}_{2}^{0} + {A}_{2} {e}^{{R}_{2} x} \qquad ( 17 )$$

where y₂⁰=0.339 IPa, A₂=−0391 kPa, and R₂=−0.0856.

When the alginate concentration is 2.5%, the fitting equation is:

$$y = {y}_{2.5}^{0} + {A}_{2.5} {e}^{{R}_{2.5} x} \qquad ( 18 )$$

where y_{2.5}⁰=1.0754 kPa, A_{2.5}=−0.946 kPa, and R_{2.5}=−0.0783.

The fitting equation for alginate concentration of 3% is:

$$y = {y}_{3}^{0} + {A}_{3} {e}^{{R}_{3} x} \qquad ( 19 )$$

where y₃⁰=2.151 kPa, A₃=−1.684 kPa, and R₃=−0.0747,

When the Ca²⁺:—COOH molar ratio is 0.18, the fitting equation is:

$$y = {y}_{0.18}^{0} + {A}_{0.18} {e}^{{R}_{0.18} x} \qquad ( 20 )$$

where y_{0.18}⁰=0.339 kPa, A_{0.18}=−0.391 kPa, and R_{0.18}=−0.0856.

The fitting equation for Ca²⁺:—COOH molar ratio of 0.27 is:

$$y = {y}_{0.27}^{0} + {A}_{0.27} {e}^{{R}_{0.27} x} \qquad ( 21 )$$

where y_{0.27}⁰=2.578 kPa. A_{0.27}=−1.865 kPa, and R_{0.27}=−0.0709.

The fitting equation for Ca²⁺:—COOH molar ratio of 0.36 is:

$$y = {y}_{0.36}^{0} + {A}_{0.36} {e}^{{R}_{0.36} x} \qquad ( 22 )$$

where y_{0.36}⁰=4.612 kPa, A_{0.36}=−3.517 kPa, and R_{0.36}=−0.0669.

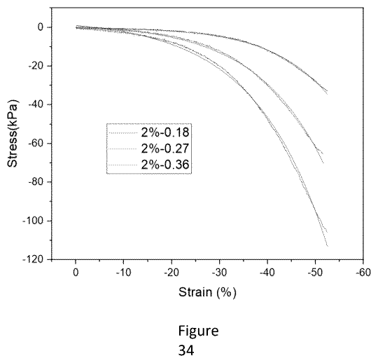

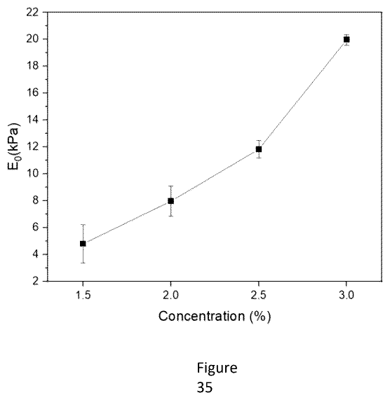

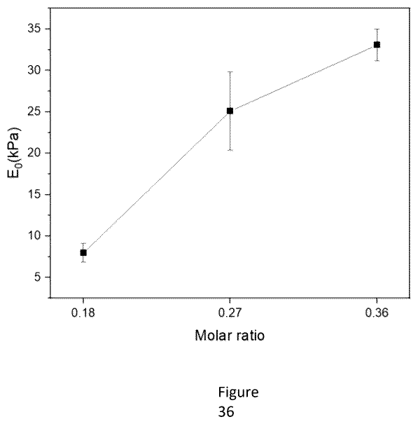

In order to compare with the tensional testing results presented earlier, E₀ of the samples was also investigated. Considering the non-linear behavior of the materials, the compressive YM within the initial 20% strain was investigated, which is within the same range as the tensional testing results presented in previous FIGS. 26 and 27. The results for the compressive tests are shown in FIGS. 35 and 36. FIG. 35 shows the compressive E₀ of the hydrogels as a function of alginate concentration. FIG. 36 shows the compressive E₀ as a function of Ca²⁺:—COOH molar ratio.

The results of FIG. 35 show that the compressive E₀ increases from 4.8 kPa to 19.97 kPa with the increase of alginate concentration from 1.5% to 3%, which is 128%-239% higher than that of the tensional test results presented in FIG. 28. FIG. 36 shows that the compressive E₀ increases from 7.97 kPa to 33.08 kPa while the molar ratio increases from 0.18 to 0.36, which is also 153%-197% higher than that obtained from the tensional tests in FIG. 29. Similarly, linear regression analysis was applied to both compressive data sets, resulting in the following relation for different alginate concentrations:

$${E}_{0}^{C} = {A}^{C} x - {B}^{C} \\ where {A}^{C} = 987.4 \frac{kPa}{g / mL} and {B}^{C} = 11.07 kPa . \qquad ( 23 )$$

The equivalent relation obtained for different Ca²⁺:—COOH molar ratios is:

$${E}_{0}^{C} = {D}^{C} x - {F}^{C} \qquad ( 24 )$$

where D^{C}=139.5 kPa and F^{C}=15.62 kPa.

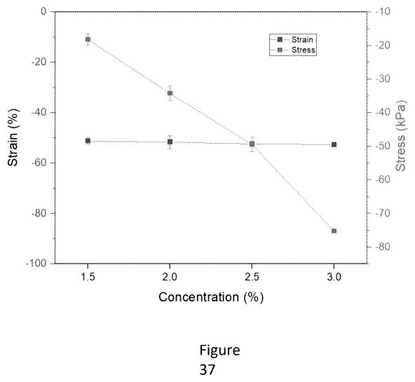

Considering the nature of the compressive testing result, the complete stress-train curve can be obtained for the prepared alginate hydrogels. Therefore, the ultimate compressive stress and strain were also investigated. FIG. 37 shows the ultimate compressive stress and strain as a function of alginate concentration. Every data point represents the average of three test results.

The results show that the ultimate compressive stress increases linearly with the increase of alginate concentration, while the ultimate strain remains stable at 50%. The ultimate stress increases from 18.15 kPa to 75.21 kPa while the alginate concentration increases from 1.5% to 3%. The following empirical relationship is obtained between the ultimate stress Eᵤ and alginate concentration for different alginate concentrations using linear regression analysis:

$${E}_{u} = {A}_{u} x - {B}_{u} \qquad ( 25 )$$

where Aᵤ=3728 kPa/g/mL and Bᵤ=39.64 kPa.

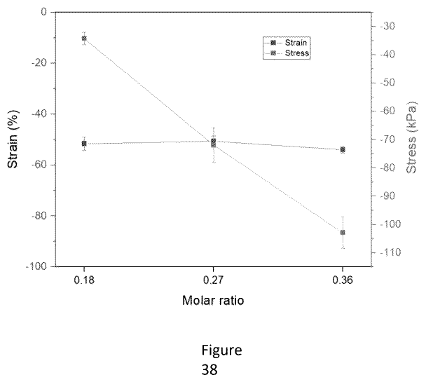

FIG. 38 shows the ultimate compressive stress and strain as a function of Ca²⁺:—COOH molar ratio, every data point represents the average of 3 test results. The results show that the ultimate compressive stress increases linearly with the increase of Ca²⁺:—COOH molar ratio, while the ultimate strain remains at 50%. The ultimate stress increases from 34.2 kPa to 102.85 kPa while the Ca²⁺:—COOH molar ratio increases from 0.18 to 0.36. By applying linear regression for different Ca²⁺:—COOH molar ratios, the following relationship is obtained:

$${E}_{u} = {D}_{u} x - {F}_{u} \qquad ( 26 )$$

where Dᵤ=381.39 kPa and Fᵤ=33.36 kPa.

The combination of the data from SEM, FTIR and mechanical testing may partly explain the observed increase in mechanical strength with the increase of alginate concentration and Ca²⁺:—COOH molar ratio. SEM images and porosity analysis show that increase of both the concentration and Ca²⁺:—COOH molar ratio will lead to lower porosity, in other words, higher 3D structural density. However, FTIR results show there is no change in molecular structure when the alginate concentration changes, but a change of molecular structure is observed when Ca²⁺:—COOH molar ratio changes. The FTIR result indicates that the change of alginate concentration only influences the density of the 3D structure, while the change of Ca²⁺:—COOH molar ratio influences both the 3D structure density and the molecular structure of the hydrogel.

### 1.7. Theoretical Calculation of Radiological Properties

Elemental composition of alginate hydrogels with different concentrations and Ca²⁺:—COOH molar ratios were calculated and compared with human lung (ICRU 44) and the results are presented in Table 4. It can be concluded from Table 4 that the elemental compositions of H, O, Na and Ca are close to those of human lung with only C having significant difference.

TABLE 4
Elemental composition of human lung and
alginate hydrogels (weight fraction)

| | ICRU | 1.5%- | 2%- | 2.5%- | 3%- | 2%- | 2%- | 5%- |
|---|---|---|---|---|---|---|---|---|
| Element | 44 | 0.18 | 0.18 | 0.18 | 0.18 | 0.27 | 0.36 | 0.18 |
| C | 0.105 | 0.007 | 0.010 | 0.012 | 0.015 | 0.011 | 0.012 | 0.024 |
| H | 0.103 | 0.110 | 0.109 | 0.109 | 0.108 | 0.109 | 0.109 | 0.106 |
| O | 0.749 | 0.881 | 0.878 | 0.876 | 0.873 | 0.877 | 0.875 | 0.863 |
| N | 0.031 | — | — | — | — | — | — | — |
| Na | 0.002 | 0.002 | 0.002 | 0.003 | 0.003 | 0.002 | 0.002 | 0.005 |
| P | 0.002 | — | — | — | — | — | — | — |
| S | 0.003 | — | — | — | — | — | — | — |
| Cl | 0.003 | — | — | — | — | — | — | — |
| K | 0.002 | — | — | — | — | — | — | — |
| Ca | — | 0.001 | 0.001 | 0.001 | 0.001 | 0.001 | 0.001 | 0.002 |

“—”: Element not present in the material.

ρₑ of alginate hydrogels with different concentrations and Ca²⁺:—COOH molar ratios were calculated and compared with human lung (ICRU 44) and the results are summarized in Table 5. For simplicity, the density of hydrogels is assumed to be 1 g/cm³:

TABLE 5
Electron densities of human lung and alginate hydrogels

| | ICRU 44 | 1.5%-0.18 | 2%-0.18 | 2.5%-0.18 | 3%-0.18 | 2%-0.27 | 2%-0.36 |
|---|---|---|---|---|---|---|---|
| ρₑ | 3.35 × 10²³ | 3.41 × 10²³ | 3.43 × 10²³ | 3.45 × 10²³ | 3.48 × 10²³ | 3.45 × 10²³ | 3.46 × 10²³ |

It can be seen from Table 5 that ρₑ of all alginate hydrogels are close to that of human lung, which indicate that ρₑ of synthesized alginate hydrogels matches that of human lung well.

Z_{eff} of alginate hydrogels are calculated and shown in Table 6. The result from Table 6 shows that all alginate hydrogels hold an outstanding Z_{eff} which is close to that of human lung.

TABLE 6
Effective atomic numbers of human lung and alginate hydrogels

| | ICRU 44 | 1.5%-0.18 | 2%-0.18 | 2.5%-0.18 | 396-0.18 | 2%-0.27 | 2%-0.36 |
|---|---|---|---|---|---|---|---|
| Z_{eff} | 7.49 | 7.44 | 7.45 | 7.46 | 7.46 | 7.46 | 7.47 |

I of alginate hydrogels is also calculated following the Bragg additivity rule. I for selected individual elements [13] is listed in Table 7:

TABLE 7
Mean excitation energy of selected elements

| | C | H | O | Na | Ca |
|---|---|---|---|---|---|
| I(eV) | 78.0 | 19.2 | 95.0 | 149.0 | 191.0 |

The calculated I of alginate hydrogels is listed in Table 8. Table 8 shows that all alginate hydrogels have a consistent and promising I which is close to that of human lung.

TABLE 8
Mean excitation energy of human lung and alginate hydrogels

| | ICRU 44 | 1.5%-0.18 | 2%-0.18 | 2.5%-0.18 | 3%-0.18 | 2%-0.27 | 2%-0.36 |
|---|---|---|---|---|---|---|---|
| I(eV) | 75.2 | 69.2 | 69.3 | 69.4 | 69.5 | 69.3 | 69.3 |

### 2. Alginate Hydrogel Foams

### 2.1. Theoretical Calculation of Radiological Properties

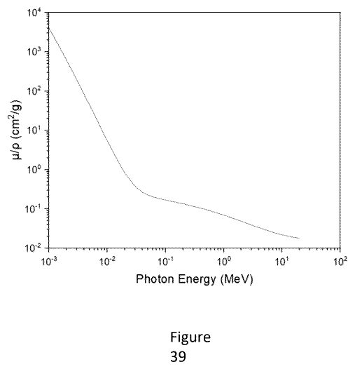

The elemental composition of alginate hydrogel with a concentration of 5% and Ca²⁺:—COOH molar ratio of 0.18 was calculated as described in Section 1.7 and the results are presented in Table 4. By following the additive rules in previous Equation (5), pp obtained for the alginate hydrogel is listed in Table 9 and plotted in FIG. 39.

TABLE 9
X-ray mass attenuation coefficients of
alginate hydrogel with concentration of 5%
and Ca²⁺:—COOH molar ratio of 0.18

| Energy | μ/ρ |
|---|---|
| (MeV) | (cm²/g) |
| 1.00E−03 | 4.03E+03 |
| 1.50E−03 | 1.37E+03 |
| 2.00E−03 | 6.17E+02 |
| 3.00E−03 | 1.93E+02 |
| 4.00E−03 | 8.28E+01 |
| 5.00E−03 | 4.35E+01 |
| 6.00E−03 | 2.52E+01 |
| 8.00E−03 | 1.06E+01 |
| 1.00E−02 | 5.48E+00 |
| 1.50E−02 | 1.72E+00 |
| 2.00E−02 | 8.30E−01 |
| 3.00E−02 | 3.81E−01 |
| 4.00E−02 | 2.70E−01 |
| 5.00E−02 | 2.27E−01 |
| 6.00E−02 | 2.06E−01 |
| 8.00E−02 | 1.83E−01 |
| 1.00E−01 | 1.70E−01 |
| 1.50E−01 | 1.50E−01 |
| 2.00E−01 | 1.36E−01 |
| 3.00E−01 | 1.18E−01 |
| 4.00E−01 | 1.06E−01 |
| 5.00E−01 | 9.64E−02 |
| 6.00E−01 | 8.91E−02 |
| 8.00E−01 | 7.83E−02 |
| 1.00E+00 | 7.04E−02 |
| 1.25E+00 | 6.29E−02 |
| 1.50E+00 | 5.72E−02 |
| 2.00E+00 | 4.92E−02 |
| 3.00E+00 | 3.95E−02 |
| 4.00E+00 | 3.39E−02 |
| 5.00E+00 | 3.02E−02 |
| 6.00E+00 | 2.76E−02 |
| 8.00E+00 | 2.42E−02 |
| 1.00E+01 | 2.21E−02 |
| 1.50E+01 | 1.94E−02 |
| 2.00E+01 | 1.81E−02 |

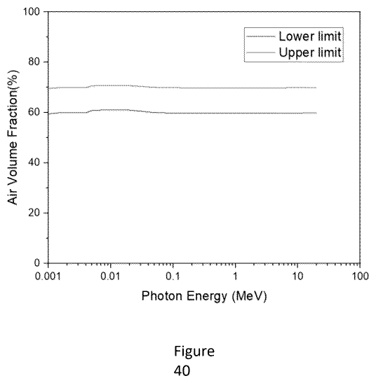

μ/ρ of air and water were obtained directly from NIST Standard Reference Database 126 [13]. Therefore, the calculated air volume ratio of the hydrogel foam for equivalent HU value between −600 to −700 of human lung is listed in Table 10 and plotted in FIG. 40.

TABLE 10
Air volume ratio range of the hydrogel foam

| Energy | Lower air | Upper Air |
|---|---|---|
| (MeV) | volume (%) | Volume (%) |
| 1.00E−03 | 59.48 | 69.61 |
| 1.5OE−03 | 59.94 | 69.95 |
| 2.00E−03 | 59.95 | 69.96 |
| 3.00E−03 | 59.97 | 69.98 |
| 4.00E−03 | 59.99 | 69.99 |
| 5.00E−03 | 60.85 | 70.64 |
| 6.00E−03 | 60.92 | 70.69 |
| 8.00E−03 | 61.03 | 70.77 |
| 1.00E−02 | 61.08 | 70.81 |
| 1.50E−02 | 61.08 | 70.81 |
| 2.00E−02 | 60.97 | 70.72 |
| 3.00E−02 | 60.56 | 70.42 |
| 4.00E−02 | 60.25 | 70.19 |
| 5.00E−02 | 60.07 | 70.05 |
| 6.00E−02 | 59.96 | 69.97 |
| 8.00E−02 | 59.86 | 69.89 |
| 1.00E−01 | 59.84 | 69.88 |
| 1.50E−01 | 59.82 | 69.86 |
| 2.00E−01 | 59.81 | 69.86 |
| 3.00E−01 | 59.81 | 69.86 |
| 4.00E−01 | 59.81 | 69.85 |
| 5.00E−01 | 59.79 | 69.84 |
| 6.00E−01 | 59.79 | 69.84 |
| 8.00E−01 | 59.80 | 69.85 |
| 1.00E+00 | 59.79 | 69.84 |
| 1.25E+00 | 59.79 | 69.84 |
| 1.50E+00 | 59.79 | 69.84 |
| 2.00E+00 | 59.79 | 69.84 |
| 3.00E+00 | 59.80 | 69.85 |
| 4.00E+00 | 59.80 | 69.85 |
| 5.00E+00 | 59.82 | 69.86 |
| 6.00E+00 | 59.82 | 69.87 |
| 8.00E+00 | 59.84 | 69.88 |
| 1.00E+01 | 59.86 | 69.90 |
| 1.50E+01 | 59.90 | 69.92 |
| 2.00E+01 | 59.91 | 69.94 |

The air volume ratio from theoretical calculation stabilizes at 60%-70% for the energy range from 1×10⁻³ to 2×10¹ MeV, which demonstrates a promising potential for application of the hydrogel foam for subsequent manufacturing processes.

### 2.2. Morphology

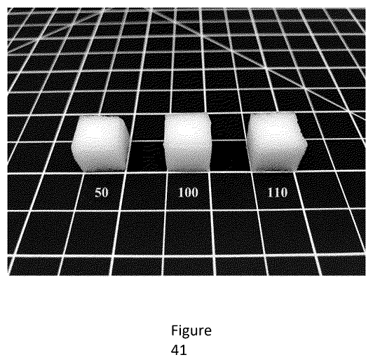

FIG. 41 shows the samples of alginate hydrogel foams produced. All samples were cut into cubes with a side length of 12.7 mm. The results show that homogeneous, three-dimensionally stable hydrogel foams are formed. There is no apparent difference in the physical appearance of the hydrogel foams irrespective of the air volume ratio. The free-standing of the hydrogel foams indicates that the network formed inside the hydrogel is strong enough to support the weight of the foam itself. The foam also demonstrates a time stability over a few weeks except for a small amount of water seeping out of the foam body.

### 2.3. Density

ρ of the hydrogel foams prepared was calculated by measuring the mass and volume of the foam samples and the results are presented in Table 11. The results indicate difference in p for samples with different air volume ratios. The lowest density of 0.482 g/cm³ is achieved for sample 50 which mixed with sufficient air. Then with the decrease of air volume mixed, the density increased to 0.685 g/cm³.

TABLE 11
Density of alginate hydrogel foams

| | Samples | | |
|---|---|---|---|
| | 50 | 100 | 110 |
| Density | 0.482 | 0.618 | 0.685 |
| (g/cm³) | | | |
| Theoretical | — | 0.714 | 0.786 |
| density | | | |
| (g/cm³) | | | |

The theoretical ρ was also calculated through the controlled air volume ratio. The measured ρ is observed to be 0.1 g/cm³ smaller than that obtained from theoretical calculations. This trend can be explained by the expansion of hydrogel due to the internal stress generated during gelling process, which will cause a decrease in ρ. A few gaps observed within the hydrogel foams in the CT images shown in Section 2.6 can also validate this explanation.

### 2.4. Optical Microscopy

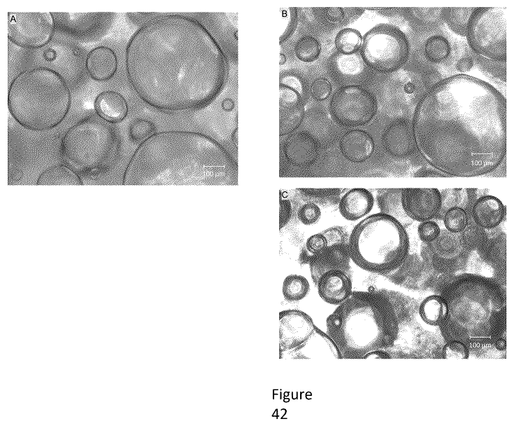

The optical microscopy images of hydrogel foam samples with different air volume ratios are shown in FIG. 42.

FIG. 42 shows the optical structure of hydrogel foams with clear images of pores of different diameters. The number of pores and diameter distribution were calculated using ImageJ. Six images were taken for each sample, the dimension for each image was 1125.27 μm×843.96 μm, giving the total analyzed area for each sample of 5.7 mm².

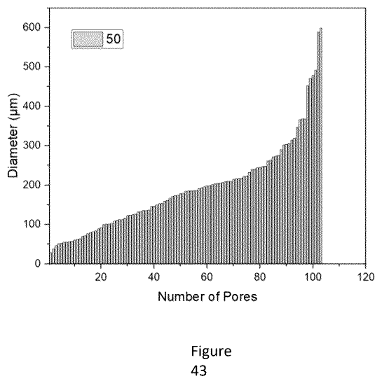

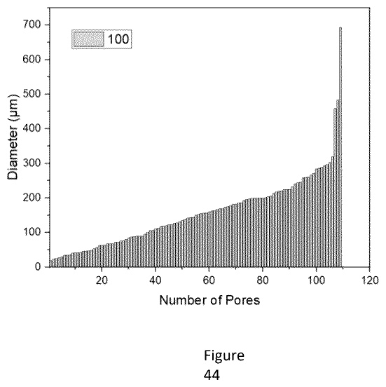

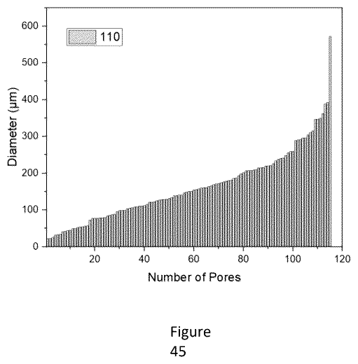

FIGS. 43, 44 and 45 show the number of pores and pore diameter distributions of hydrogel foams corresponding to sample 50, 100 and 110, respectively, indicating different air volume ratios.

FIGS. 43 to 45 show that the pore diameter is approximately evenly distributed from tens of micrometers to about 500 micrometers. The diameter of pores slightly decreases with the decrease of volume of air. However, the number of pores exhibits a trend of increasing from 103 to 115 within the area characterized.

### 2.5. Mechanical Properties

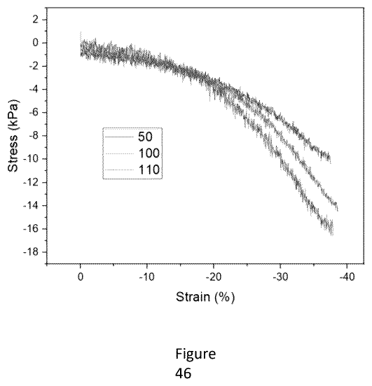

FIG. 46 shows the compressive stress-strain relations of the prepared hydrogel foams with different air volume ratios. The results indicate that all the samples have similar non-linear stress-strain behaviors. For every individual sample, the stress increases faster with the increase of strain.

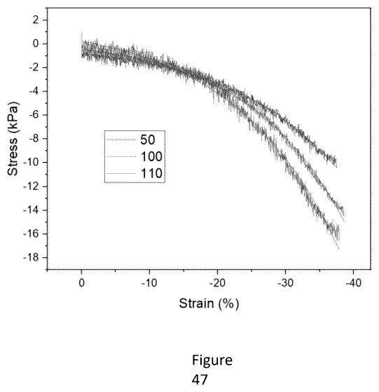

Exponential curve fitting was conducted for the compressive stress-strain relation of the hydrogel foams. Applying Exponential function to the hydrogel foams as the fitting model, the results are shown in FIG. 47.

The fitting equations for alginate hydrogel foams for different air volume ratios are as follows:

For sample 50, the fitting equation is:

$$y = {y}_{50}^{0} + {A}_{50} {e}^{{R}_{50} x} \qquad ( 27 )$$

where y₅₀⁰=0.227 kPa, A₅₀=−1.029 kPa and R₅₀=−0.0624.

For sample 100, the fitting equation is:

$$y = {y}_{100}^{0} + {A}_{100} {e}^{{R}_{100} x} \qquad ( 28 )$$

where y₁₀₀⁰=0.562 kPa, A₂₀₀=−1.007 kPa and R₁₀₀=−0.0709.

For sample 110, the fitting equation is:

$$y = {y}_{110}^{0} + {A}_{110} {e}^{{R}_{110} x} \qquad ( 29 )$$

where y₁₁₀⁰=1.874 kPa, A₁₁₀=−1.649 kPa and R₁₁₀=−0.0649,

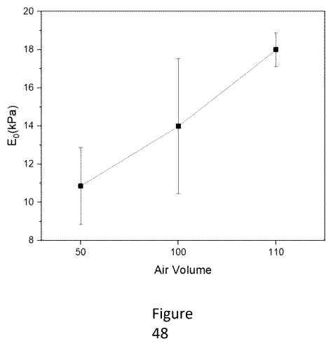

The compressive E₀ of the samples was investigated as well for hydrogel foams of different air volume ratios. Similar to the analysis for hydrogels, the compressive YM was investigated within the initial 20% strain. The results for the compressive tests are shown in FIG. 48.

The results of FIG. 48 show that the compressive E₀ of alginate hydrogel foams increases from 10.86 kPa to 18 kPa when air volume ratio decreases from sample 50 to sample 110.

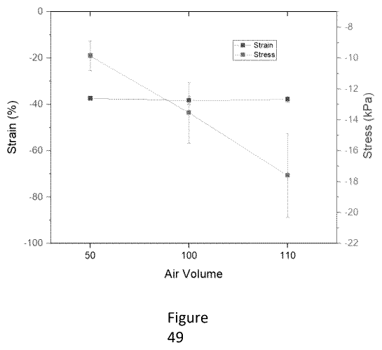

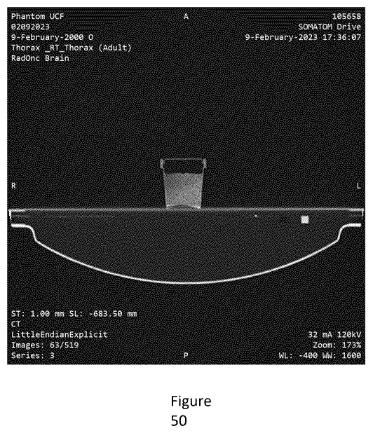

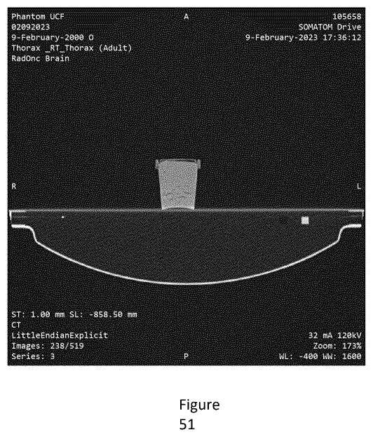

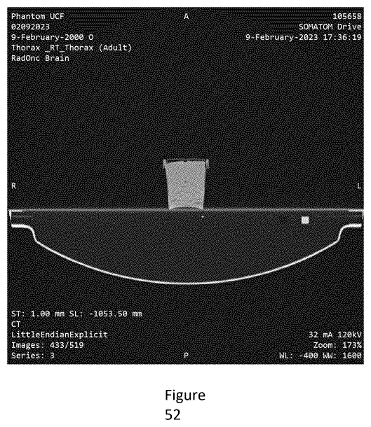

Similarly, the complete stress-strain curve was obtained for the hydrogel foams, as well as the ultimate compressive stress and strain. FIG. 49 shows the ultimate compressive stress and strain of the alginate hydrogel foams. Every data point represents the average of three test results.

The results show that the ultimate compressive stress increases from 9.84 kPa to 17.58 kPa with the decrease of air volume ratio. While at the same time, the ultimate strains remain stable at approximately 38%.

### 2.6. Radiological Properties

FIGS. 47, 48 and 49 show the CT scan images of hydrogel foams with different air volume ratios corresponding to samples 50, 100 and 110, respectively.

The images obtained show that mostly homogenous hydrogel foams were formed. A few gaps were observed within the samples of 100 and 110. The formation of those gaps may be due to the internal stress generated within the hydrogel during gelling process. The HU values of five cross-sections for each sample were calculated and presented in Table 12 with the corresponding theoretical calculation result.

TABLE 12
HU values of alginate hydrogel foams

| | | | | | | | | Calculated |
|---|---|---|---|---|---|---|---|---|
| | | | | | | | | HU based |
| | HU-1 | HU-2 | HU-3 | HU-4 | HU-5 | Average | StDev | on density |
| 50 | −468.03 | −458.58 | −463.71 | −454.32 | −455.22 | −459.97 | 5.82 | −504.57 |
| 100 | −292.40 | −294.77 | −297.51 | −304.22 | −298.00 | −297.38 | 4.44 | −364.78 |
| 110 | −230.75 | −233.18 | −232.62 | −235.10 | −232.84 | −232.90 | 1.55 | −295.91 |

Table 12 shows that various HU values were obtained for samples with different air volume ratios. By comparing with theoretical calculation results based on the measured ρ of the foams, it can be concluded that the HU values obtained from CT scan is generally close to that obtained from theoretical calculation, which in turn validated the result of air volume ratio calculated in

### LIST OF ABBREVIATIONS/ACRONYMS

ρ Density
YM Young's modulus
ρₑ Electron density
Z_{eff} Effective atomic number
I Mean excitation energy
μ/ρ Mass attenuation coefficient
μ Linear attenuation coefficient
ρ Density
HU Hounsfield unit
CT Computed tomography
PU Polyurethan
ECM Extra cellular matrix
CaCO₃ Calcium carbonate
GDL D-(+)-gluconic acid δ-lactone
DI Deionized
[η] Intrinsic viscosity
SEM Scanning electron microscopy
CPD Critical point drying
FTIR Fourier transform infrared
E₀ Initial stage Young's modulus
SLES 70 Sodium lauryl ether sulfate 70%
Eᵤ Ultimate stress

### REFERENCES

[1] N. N. C. Institute, Types of Cancer Treatment.
[2] P. J. Keall, G. S. Mageras, J. M. Balter, R. S. Emery, K. M. Forster, S. B. Jiang, J. M. Kapatoes, D. A. Low, M. J. Murphy, B. R. J. M.p. Murray, The management of respiratory motion in radiation oncology report of AAPM Task Group 76 a, 33(10) (2006) 3874-3900.
[3] A. Al-Mayah, J. Moseley, K. K. Brock, Contact surface and material nonlinearity modeling of human lungs, Physics in Medicine and Biology 53(1) (2008) 305-317.
[4] X. Wang, X. Zhang, X. Dai, X. Wang, X. Li, J. Diao, T. Xu, Tumor-like lung cancer model based on 3D bioprinting, 3 Biotech 8(12) (2018) 501.
[5] M. Li, E. Castillo, X. L. Zheng, H. Y. Luo, R. Castillo, Y. Wu, T. Guerrero, Modeling lung deformation: A combined deformable image registration method with spatially varying Young's modulus estimates, Medical physics 40(8) (2013).
[6] B. Grosswendt, ICRP publication 110, Oxford University Press, 2012.
[7] A. P. Santhanam, Y. Min, S. P. Mudur, A. Rastogi, B. H. Ruddy, A. Shah, E. Divo, A. Kassab, J. P. Rolland, P. Kupelian, An inverse hyper-spherical harmonics-based formulation for reconstructing 3D volumetric lung deformations, Comptes Rendus Mécanique 338(7-8) (2010) 461-473.
[8] S. J. Lai-Fook, R. E. Hyatt, Effects of age on elastic moduli of human lungs, Journal of Applied Physiology 89(1) (2000) 163-168.
[9] B. Goss, K. McGee, S. Kruse, A. Manduca, R. Ehman, Magnetic resonance elastography of the lung: Initial feasibility, Proceedings of International Society of Magnetic Resonance in Medicine, 2003, p. 429.
[10] F. Liu, B. S. Shea, J. D. Mih, A. M. Tager, D. J. J. B. J. Tschumperlin, Lung parenchymal tissue stiffness in fibrosis and cellular responses to substrate stiffness, 96(3) (2009) 395a.
[11] O. J. Ilegbusi, B. Seyfi, R. Salvin, Patient-specific model of lung deformation using spatially dependent constitutive parameters, Mathematical and Computer Modelling of Dynamical Systems 20(6) (2014) 546-556.
[12] R. Murty, Effective atomic numbers of heterogeneous materials, Nature 207(4995) (1965) 398-399.
[13] J. Hubbell, S. Seltzer, X-Ray mass attenuation coefficients: NIST standard reference database 126, National Institute of Standards and Technology, Gaithersburg, Maryland, USA (2004).
[14] E. A. Kazerooni, B. H. Gross, Cardiopulmonary imaging, Lippincott Williams & Wilkins 2004.
[15] Density and mass of each organ/tissue.
[16] R. Griffith, P. Dean, A. Anderson, J. Fisher, A tissue-equivalent torso phantom. For intercalibration of in-vivo, transuranic-nuclide counting facilities, Advances in radiation protection monitoring 1979.
[17] F. Y. Taylor, History of the Lawrence Livermore National Laboratory Torso Phantom, (1997).
[18] R. J. Traub, P. C. Olsen, J. C. McDonald, The radiological properties of a novel lung tissue substitute, Radiation protection dosimetry 121(2) (2006) 202-207.
[19] A. M. Ferreira, P. Gentile, V. Chiono, G. J. A.b. Ciardelli, Collagen for bone tissue regeneration, 8(9) (2012) 3191-3200.
[20] P. J. Pannone, Trends in biomaterials research, Nova Publishers 2007.
[21] M. Hospodiuk, M. Dey, D. Sosnoski, I. T. J. B.a. Ozbolat, The bioink: a comprehensive review on bioprintable materials, 35(2) (2017) 217-239.
[22] L. Pescosolido, W. Schuurman, J. Malda, P. Matricardi, F. Alhaique, T. Coviello, P. R. van Weeren, W. J. Dhert, W. E. Hennink, T. J. B. Vermonden, Hyaluronic acid and dextran-based semi-IPN hydrogels as biomaterials for bioprinting, 12(5) (2011) 1831-1838.
[23] M. C. Gómez-Guillén, J. Turnay, M. Fernindez-Diaz, N. Ulmo, M. A. Lizarbe, P. J. F. H. Montero, Structural and physical properties of gelatin extracted from different marine species: a comparative study, 16(1) (2002) 25-34.
[24] J. H. Chung, S. Naficy, Z. Yue, R. Kapsa, A. Quigley, S. E. Moulton, G. G. J. B. S. Wallace, Bio-ink properties and printability for extrusion printing living cells, 1(7) (2013) 763-773.
[25] M. J. P.i.p.s. Rinaudo, Chitin and chitosan: properties and applications, 31(7) (2006) 603-632.
[26] F. Croisier, C. J. E.p.j. Jérôme, Chitosan-based biomaterials for tissue engineering, 49(4) (2013) 780-792.
[27] F. J. Pavinatto, L. Caseli, O. N. J. B. Oliveira Jr, Chitosan in nanostructured thin films, 11(8) (2010) 1897-1908.
[28] S. V. Madihally, H. W. J. B. Matthew, Porous chitosan scaffolds for tissue engineering, 20(12) (1999) 1133-1142.
[29] F. A. JOHNSON, D. Q. CRAIG, A. D. J. J.o.p. MERCER, pharmacology, Characterization of the block structure and molecular weight of sodium alginates, 49(7) (1997) 639-643.
[30] S. Liu, H. Li, B. Tang, S. Bi, L. J. C.p. Li, Scaling law and microstructure of alginate hydrogel, 135 (2016) 101-109.
[31] K. Y. Lee, D. J. J. P.i.p.s. Mooney, Alginate: properties and biomedical applications, 37(1) (2012) 106-126.
[32] P. J. C.p. Gacesa, Alginates, 8(3) (1988) 161-182.
[33] J. C. Sun, H. P. Tan, Alginate-Based Biomaterials for Regenerative Medicine Applications, Materials 6(4) (2013) 1285-1309.
[34] G. T. Grant, E. R. Morris, D. A. Rees, P. J. C. Smith, D. Thom, BIOLOGICAL INTERACTIONS BETWEEN POLYSACCHARIDES AND DIVALENT CATIONS—EGGBOX MODEL, Febs Letters 32(1) (1973) 195-198.
[35] J. L. Drury, R. G. Dennis, D. J. Mooney, The tensile properties of alginate hydrogels, Biomaterials 25(16) (2004) 3187-3199.
[36] A. D. Augst, H. J. Kong, D. J. Mooney, Alginate hydrogels as biomaterials, Macromolecular bioscience 6(8) (2006) 623-633.
[37] C. K. Kuo, P. X. Ma, Ionically crosslinked alginate hydrogels as scaffolds for tissue engineering: Part 1. Structure, gelation rate and mechanical properties, Biomaterials 22(6) (2001) 511-521.
[38] J. Venkatesan, I. Bhatnagar, P. Manivasagan, K. H. Kang, S. K. Kim, Alginate composites for bone tissue engineering: A review, International Journal of Biological Macromolecules 72 (2015) 269-281.
[39] S. J. Liu, A. K. Bastola, L. Li, A 3D Printable and Mechanically Robust Hydrogel Based on Alginate and Graphene Oxide, Acs Applied Materials & Interfaces 9(47) (2017) 41473-41481.
[40] A. Kosik-Koziol, M. Costantini, T. Bolek, K. Szoke, A. Barbetta, J. Brinchmann, W. Swieszkowski, PLA short sub-micron fiber reinforcement of 3D bioprinted alginate constructs for cartilage regeneration, Biofabrication 9(4) (2017).
[41] A. G. Tabriz, M. A. Hermida, N. R. Leslie, W. M. Shu, Three-dimensional bioprinting of complex cell laden alginate hydrogel structures, Biofabrication 7(4) (2015).
[42] F. E. Freeman, D. J. Kelly, Tuning Alginate Bioink Stiffness and Composition for Controlled Growth Factor Delivery and to Spatially Direct MSC Fate within Bioprinted Tissues, Scientific Reports 7 (2017).
[43] O. Jeon, K. H. Bouhadir, J. M. Mansour, E. Alsberg, Photocrosslinked alginate hydrogels with tunable biodegradation rates and mechanical properties, Biomaterials 30(14) (2009) 2724-2734.
[44] C. K. Kuo, P. X. Ma, Maintaining dimensions and mechanical properties of ionically crosslinked alginate hydrogel scaffolds in vitro, Journal of Biomedical Materials Research Part A: An Official Journal of The Society for Biomaterials, The Japanese Society for Biomaterials, and The Australian Society for Biomaterials and the Korean Society for Biomaterials 84(4) (2008) 899-907.
[45] S. Naghieh, M. R. Karamooz-Ravari, M. Sarker, E. Karki, X. J. J.o.t.m.b.o.b.m. Chen, Influence of crosslinking on the mechanical behavior of 3D printed alginate scaffolds: Experimental and numerical approaches, 80 (2018) 111-118.
[46] A. Jones, D. Hintenlang, W. Bolch, Tissue—equivalent materials for construction of tomographic dosimetry phantoms in pediatric radiology, Medical physics 30(8) (2003) 2072-2081.
[47] R. Kienbock, On the quantimetric method, Archives of the Roentgen Ray 11(1) (1906) 17-20.
[48] K. Goldstone, Tissue Substitutes in Radiation Dosimetry and Measurement, in: ICRU Report 44, International Commission on Radiation Units and Measurements, USA (1989), WB Saunders, 1990.
[49] R. J. Traub, P. C. Olsen, J. C. J. R.p.d. McDonald, The radiological properties of a novel lung tissue substitute, 121(2) (2005) 202-207.
[50] A. El-Khayatt, A. Ali, V. P. Singh, N. J. R. E. Badiger, D.i. Solids, Determination of mass attenuation coefficient of low-Z dosimetric materials, 169(12) (2014) 1038-1044.
[51] R. J. Traub, Influence of manufacturing processes on the performance of phantom lungs, Pacific Northwest National Lab. (PNNL), Richland, WA (United States), 2008.
[52] M. Berger, J. Hubbell, 1999. XCOM: Photon Cross-Sections Database, Web Version 1.2, National Institute of Standards and Technology, Gaithersburg, MD20899, USA, 1987.
[53] A. Martinsen, G. Skjåk-Bræk, O. Smidsrod, F. Zanetti, S. Paoletti, Comparison of different methods for determination of molecular weight and molecular weight distribution of alginates, Carbohydrate polymers 15(2) (1991) 171-193.
[54] O. J. C.r. Smidsrod, Solution properties of alginate, 13(3) (1970) 359-372.
[55] B. Nordestgaard, J. Rostgaard, Critical-point drying versus freeze drying for scanning electron microscopy: A quantitative and qualitative study on isolated hepatocytes, Journal of microscopy 137(2) (1985) 189-207.
[56] P. Salemink, Damage of cells by cryopreservation, Comparative Haematology International 1 (1991) 208-213.
[57] S. D. Allison, T. W. Randolph, M. C. Manning, K. Middleton, A. Davis, J. F. Carpenter, Effects of drying methods and additives on structure and function of actin: mechanisms of dehydration-induced damage and its inhibition, Archives of Biochemistry and Biophysics 358(1) (1998) 171-181.
[58] I. Khan, A. Elhissi, M. Shah, M. A. Alhnan, W. Ahmed, Liposome-based carrier systems and devices used for pulmonary drug delivery, Biomaterials and medical tribology, Elsevier 2013, pp. 395-443.
[59] Supercritical carbon dioxide.
[60] Carbon dioxide pressure-temperature phase diagram, Wikimedia Commons, the free media repository, 2010.
[61] M. Oyen, Mechanical characterisation of hydrogel materials, International Materials Reviews 59(1) (2014) 44-59.
[62] J. A. Hinkley, L. D. Morgret, S. H. Gehrke, Tensile properties of two responsive hydrogels, Polymer 45(26) (2004) 8837-8843.

What is claimed is:

1. Alginate hydrogel comprised of sodium alginate, CaCO₃, and D-(+)-gluconic acid δ-lactone (GDL), wherein, optionally, the GDL has a MW of about 178.

2. The Alginate hydrogel of claim 1, wherein the alginate hydrogel is made by a method comprising the steps of:
i. dissolving sodium alginate in deionized water and continuously stirring the solution until homogeneous transparent to light yellow solution is obtained,
ii. applying vacuum to the alginate solution to remove dissolved gas, wherein applying optionally comprises placing the alginate solution in a vacuum chamber at approx. 85 kPa for approx. 24 hours to remove dissolved gas,
iii. admixing components into the alginate solution to form a mixture as follows:
a. adding CaCO₃ to the alginate solution, and mixing the mixture for about 1 min, and
b. adding GDL to the mixture and mixing the mixture for about 1 min, and
iv. sealing the mixture to gel for approx. 24 hours to form homogeneous hydrogels.

3. The alginate hydrogel of claim 1, wherein alginate concentration (weight/volume) is between about 0.5% and about 5%, optionally 1.5%, 2%, 2.5%, or 3%, and wherein Ca²⁺:—COOH molar ratio is between about 0.15 and about 0.4, optionally 0.18, 0.27, or 0.36.

4. The alginate hydrogel of claim 1, wherein CaCO₃:GDL molar ratio is approximately 0.5.

5. The alginate hydrogel of claim 1, wherein the intrinsic viscosity of sodium alginate solution is in the range of 0.88-1.06 mL/g depending on the testing speed, and the molecular weight of sodium alginate is in the range of about 4.4×10⁴ to about 5.3×10⁴ g/mol.

6. The alginate hydrogel of claim 1, wherein the hydrogel has homogeneous, transparent, and three-dimensionally stable network structure and time stability over a duration of several weeks.

7. The alginate hydrogel of claim 1, wherein the density of the hydrogel is approximately 1 g/cm³.

8. The alginate hydrogel of claim 1, wherein the hydrogel has a highly porous structure and uniformity, wherein the pore size ranges from tens to hundreds of nanometers, and wherein the porosity ranges from about 40% to about 50%.

9. The alginate hydrogel of claim 1, wherein in FTIR spectra, the hydrogel exhibits an absorption peak between 3600-2800 cm⁻¹ combined with an absorpotion peak at 2920 cm⁻¹, which are attributed to the stretching vibration of O—H and C—H, adsorption peaks at 1594 cm⁻¹ and 1406 cm⁻¹, which are attributed to asymmetric stretching vibration of COO⁻, and absorption peaks at 1081 cm⁻¹ and 1124 cm⁻¹, which are attributed to the C—O stretching vibration when the alginate concentration is between 0.5% and 5%, optionally 1.5%, 2%, 2.5%, or 3%, and the Ca²⁺:—COOH molar ratio is about 0.18, and wherein as Ca²⁺:—COOH molar ratio increases from about 0.18 or about 0.27 to about 0.36, and the peak location of —COOH shifts from 1591-1592 cm⁻¹, 1588-1589 cm⁻¹ to 1586-1587 cm⁻¹.

10. The alginate hydrogel of claim 1, wherein the initial stage Young's modulus (E₀) of tensional stress of the hydrogel ranges from about 2 kPa to about 25 kPa depending on the alginate concentration and the Ca2+:—COOH molar ratio.

11. The alginate hydrogel of claim 1, wherein the initial stage Young's modulus (E₀) of compressive stress of the hydrogel ranges from about 4 kPa to about 35 kPa depending on the alginate concentration and the Ca2+:—COOH molar ratio.

12. The alginate hydrogel of claim 1, wherein the ultimate compressive stress (Eᵤ) of the hydrogel increases from about 18.15 kPa to about 75.21 kPa as the alginate concentration increases from about 1.5% to about 3%, while the ultimate strain of the hydrogel remains stable at about 50%, and wherein the ultimate compressive stress (Eᵤ) of the hydrogel increases from about 34.2 kPa to about 102.85 kPa as the Ca²⁺:—COOH molar ratio increases from about 0.18 to about 0.36, while the ultimate strain of the hydrogel remains at about 50%.

13. The alginate hydrogel of claim 1, wherein the elemental compositions of H, O, Na and Ca in the hydrogel are within 30% or 20% similarity to those of human lung, but wherein C is about 0.005 to about 0.0030 wt. percent.

14. The alginate hydrogel of claim 1, wherein electron density (ρₑ) of the hydrogel (3.40×10²³−3.50×10²³ g/cm³) is within 30% or 20% similarity to that of a human lung, wherein the electron density is, optionally, about 3.35×10²³ g/cm³.

15. The alginate hydrogel of claim 1, wherein effective atomic number (Z_{eff}) of the hydrogel (7.4-7.5) is within 30% or 20% similarity to that of a human lung, wherein the effective atomic number is, optionally, about 7.49.

16. The alginate hydrogel of claim 1, wherein mean excitation energy (I) of the hydrogel (69 eV-70 eV) is within 30 or 20% similarity with that of a human lung approx., wherein the mean excitation energy is about 75.2 eV.

17. Alginate hydrogel foam made of sodium alginate, CaCO₃ (MW 100.09), D-(+)-gluconic acid δ-lactone (GDL), wherein, optionally, the GDL has a MW of approximately 178, and sodium lauryl ether sulfate.

18. The alginate hydrogel foam of claim 17, wherein the alginate hydrogel is produced by a method comprising the steps of;
i. dissolving sodium alginate in deionized water to produce an alginate solution and continuously stirring the solution until homogeneous transparent to light yellow solution is obtained, and
ii. admixing components with the alginate solution to form a mixture as follows:
a. adding sodium lauryl ether sulfate into the alginate solution and stirring the mixture until fully mixed,
b. adding CaCO₃ to the mixture and continuously stirring it until the mixture is fully mixed and the color of the mixture becomes white,
c. adding GDL to the mixture to initiate gelation while mechanically mixing the mixture during the entire process, and
d. stopping mechanical mixing when the volume of the mixture no longer changes or when the mixture fills the entire space of the container.

19. The alginate hydrogel foam of claim 17, wherein alginate concentration (weight/volume) is between about 0.5% and about 5%, optionally 5%, and wherein Ca²⁺:—COOH molar ratio is between about 0.15 and about 0.4, optionally 0.18.

20. (canceled)

21. (canceled)

22. (canceled)

23. (canceled)

24. (canceled)

25. (canceled)

26. (canceled)

27. (canceled)

28. (canceled)

29. A phantom insert made of the alginate hydrogel foam of claim 18 for use with an anthropomorphic phantom that comprises an outer shell, an anatomical shape and an inner space that accommodates the phantom insert, wherein the shell is fillable with water, saline or artificial interstitial fluid, wherein the phantom insert comprises (i) a size, dimension, and shape according to a radiation therapy target organ or tissue; (ii) an anatomical shape of a human lung or kidney; (iii) is configured to hold a radiochromic film; (iv).

30. (canceled)

31. (canceled)

32. (canceled)

33. (canceled)
